(19) United States
(12) Patent Application Publication
(10) Pub. No.: US 2009/0226415 A1
(43) Pub. Date: Sep. 10, 2009

(54) MODIFIED TRIDEGINS, PRODUCTION AND USE THEREOF AS TRANSGLUTAMINASE INHIBITORS

(76) Inventors: Helmut Giersiefen, Starnberg (DE); Johannes Stoeckel, Munich (DE); Tanja Pamp, Munich (DE); Marion Ohlmann, Munich (DE)

Correspondence Address: CLARK & ELBING LLP, 101 FEDERAL STREET, BOSTON, MA, 02110, US

(21) Appl. No.: 12/406,745

(22) Filed: Mar. 18, 2009

Related U.S. Application Data
(63) Continuation of application No. 10/498,752, filed on Feb. 10, 2005, now abandoned.

(30) Foreign Application Priority Data
Dec. 21, 2001 (DE) 101 63 333.5
Dec. 12, 2002 (DE) 102 58 159.2

(51) Int. Cl. A61K 38/17; C07K 14/00; A61K 38/49; A61P 7/02

(52) U.S. Cl. 424/94.64; 530/324; 514/12

(57) ABSTRACT

The invention relates to modified tridegins, polypeptides derived from SEQ ID No. 1, in which the modification is replacement of a cysteine residue and/or one of the following amino acids—Lys2, Lys7, His10, Gly12, Leu24, Tyr31, Phe34, Arg39, Ile45, Met48, Asp50, Pro55, Phe58, Asn60, Pro65, Arg66, by another amino acid and/or N- or C-terminal deletion, whereby the remaining polypeptide comprises at least the amino acid sequence DDIYQRXVXFPXLPL (SEQ ID NO.89) and/or a covalent bonding to polyethylene glycol. Said polypeptides are novel inhibitors of transglutaminases, in particular of Factor XIIIa, of the terminal enzymes in the blood coagulation cascade. The invention further relates to methods for production of said inhibitors and the use thereof as transglutaminase inhibitors.

### CROSS REFERENCE TO RELATED APPLICATIONS

This application is a continuation of U.S. application Ser. No. 10/498,752, filed Feb. 10, 2005, which is the U.S. National Stage Application of International Application PCT/EP2002/14684, filed Dec. 20, 2002, which claims benefit of German patent applications 10258159.2 and 1063333.5, filed Dec. 12, 2002 and Dec. 21, 2001, respectively.

The present invention relates to modified tridegins, polypeptides derived from SEQ ID No. 1, with the modification consisting in at least one cysteine residue and/or one of the following amino acids—Lys2, Lys7, His10, Gly12, Leu24, Tyr31, Phe34, Arg39, Ile45, Met48, Asp50, Pro55, Phe58, Asn60, Pro65 and Arg66—being replaced by another amino acid, and/or N and C termini being deleted, with the remaining polypeptide containing at least the amino acid sequence DDIYQRXVXFPXLPL (SEQ ID No. 89), and/or there being a covalent linkage with polyethylene glycol. The polypeptides according to the invention are novel inhibitors of transglutaminases, in particular of factor XIIIa, the terminal enzyme of the blood coagulation cascade. The present invention also relates to processes for preparing these inhibitors and to the use of the latter as transglutaminase inhibitors.

Transglutaminases (EC 2.3.2.13) catalyze the formation of amide bonds within a polypeptide chain, or between different polypeptide chains, in accordance with the following reaction scheme:

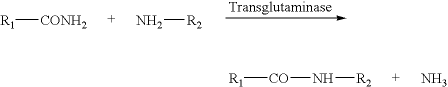

They consequently catalyze the crosslinking of proteins, by forming γ-glutamyl-ε-lysine bonds between two polypeptide chains, and thereby contribute to stabilizing many protein aggregates.

Factor XIIIa is a transglutaminase which is of great clinical importance. Factor XIIIa is the terminal enzyme in the blood coagulation cascade and covalently cross-links, by means of transglutamination, fibrin polymers in a “soft” blood thrombus. In addition, factor XIIIa is responsible for covalently bonding α₂-antiplasmin, by means of transglutamination, to the fibrin network. Such a crosslinked and modified blood thrombus is described as being a “hard” blood thrombus and cannot be broken down so rapidly by fibrinolytic enzymes as can a “soft” blood thrombus, composed of fibrin polymers without any covalent crosslinking. Consequently, factor XIIIa makes a crucial contribution to stabilizing a blood thrombus.

Inhibitors of factor XIIIa prevent the crosslinking reaction between the different chains of the fibrin network, and also the covalent bonding of α₂-antiplasmin, and thereby facilitate the prophylactic treatment of thrombotic events as well as thrombolytic treatment.

Several inhibitors of transglutaminases have already been described in the prior art (a selection is given in Table 1). These inhibitors are
immunoglobulins which bind to transglutaminases,
low molecular weight chemical compounds which react with cysteines,
low molecular weight amines which compete with natural substrates, and
active fractions isolated from leeches belonging to the species Haementeria ghilianii.

TABLE 1
Selection of published and patented inhibitors of factor XIIIa

| Inhibitor | Affinity (IC₅₀) | | Reference |
|---|---|---|---|
| Cerulenin | 29 | μM | U.S. Pat. No. 5,710,174 |
| ZG-1400 | 5.7 | μM | U.S. Pat. No. 5,710,174 |
| Imidazole compounds | >80 | nM | U.S. Pat. No. 4,968,713 |
| 2-(1-Acetonylthio)-5-methyl- | unknown | | |
| thiazolo | | | |
| [2,3-b]1,3,4-Thiadiazolium | | | |
| perchlorate | | | |
| (L-722,151) | | | |
| Monodansyl cadaverine | unknown | | U.S. Pat. No. 5,124,358 |
| Isothiocyanates | | | WO 9213530 |
| Active fraction (tridegin?) | 3.4 | nM | U.S. Pat. No. 6,025,330 |

Immunoglobulins which are directed against factor XIII have been disclosed, for example, in U.S. Pat. No. 5,470,957. In this publication, monoclonal antibodies were prepared against a subunit of factor XIIIa and it was observed that these antibodies inhibited the activation of factor XIII by thrombin. However, extensive modifications, such as preparing human chimeras, are normally required if these antibodies are to be used thera-peutically.

Another class of inhibitors consists of low molecular weight, reactive chemical compounds which bind irreversibly to the factor XIIIa active center, i.e. a cysteine residue. However, such compounds, disclosed in WO 92/13530, suffer from the disadvantage that they are very reactive and are relatively unstable in vivo. They also react with cysteine residues in other proteins and are consequently not specific for factor XIIIa. They are consequently not used as pharmaceutical active compounds.

Further transglutaminase inhibitors are low molecular weight amines, as disclosed in WO 91/10427, which act as competitive substrates for the transglutaminase reaction. However, they are consumed by the trans-glutamination reaction and alter the functionality of the proteins, to which they are coupled as a result of the transglutaminase reaction, in an unpredictable manner. In addition, they have to be used at a relatively high concentration of about 200 μM, thereby restricting their therapeutic value.

A fraction which inhibits factor XIIIa with a high degree of affinity and specificity has been isolated from the salivary gland of leeches belonging to the species Haementeria ghilianii (disclosed in U.S. Pat. No. 6,025,330). At least two different proteins were present in the purified, active fractions, with one protein having an approximate size of 7-8 kDa constituting the main protein constituent of the active fractions. The primary sequence of this polypeptide, which is 66 amino acids in length, was determined approximately using methods of protein biochemistry. This polypeptide has been named tridegin and it has been assumed that it inhibits transglutaminases in general and factor XIIIa in particular.

However, no attempt was made to separate the tridegin from the other proteins which were still present in the active fraction, which means that the possibility of it being these proteins which inhibit factor XIIIa has not been ruled out (see, in particular, Finney et al., (Finney et al., Biochem. Journal 324, 797-805 (1997), FIG. 2, lane 3). It was not even ascertained whether the factor XIIIa-inhibiting activity is peptide in nature. Consequently, other biopolymers present in the active fractions could constitute the true factor XIIIa-inhibiting activity provided they were not conspicuous during analysis on an SDS gel, for example complex sugars or lipids or glycolipids. Consequently, these publications did not demonstrate that tridegin inhibits factor XIIIa.

It is consequently completely uncertain as to whether a tridegin which was prepared recombinantly, e.g. expressed in the prokaryote Escherichia coli, would be able to function as an inhibitor of factor XIIIa. In both U.S. Pat. No. 6,025,330 (4, 12-18) and in the corresponding scientific paper (Finney et al., Biochem. Journal 324, 797-805 (1997)), it was observed that the tridegin which was purified from leeches belonging to the species Haementeria ghilianii was posttranslationally modified (Finney et al., Biochem. Journal (1997) 324, 800, right-hand column, last sentence). Although it is known that it is precisely secreted proteins, of which tridegin is also one, which frequently require such modifications for their function (e.g. described in Kemball-Cook et al., Gene, 139(2): 275-279 (1994) or Pang et al., Endocrinology 140(11): 5102-5111 (1999)), these two publications did not deal with the extent to which posttranslational modifications are required for the assumed function of tridegin. However, such modifications are lacking in proteins which have been prepared recombinantly in Escherichia coli. For this reason, it is precisely proteins which are normally located extracellularly which are frequently inactive when they are expressed in a heterologous system.

WO 49039 describes a new technique for purifying fusion proteins and, in this connection, discloses a synthetic DNA sequence which encodes tridegin. In addition, tridegin was expressed together with glucose dehydrogenase as a fusion protein, and purified; however, the activity of the fusion protein as an inhibitor of factor XIIIa was not tested.

The object of the invention is therefore to prepare novel polypeptide inhibitors of transglutaminases recombinantly or synthetically in adequate quantities and in pure form.

Surprisingly, the tridegin polypeptide which was expressed in a heterologous system, e.g. in Escherichia coli, and purified, was found to be an effective inhibitor of transglutaminase, in particular inhibitor of factor XIIIa, especially of human factor XIIIa. This thereby demonstrated, for the first time, that the tridegin polypeptide is in fact a transglutaminase inhibitor, in particular an inhibitor of factor XIIIa, especially of human factor XIIIa. Surprisingly, tridegin polypeptides possessing one or more modifications also exhibited activity as transglutaminase inhibitors, in particular as inhibitors of factor XIIIa, especially of human factor XIIIa. It was found, surprisingly, that recombinant tridegin polypeptide expressed in the yeast Pichia pastoris is an even more effective transglutaminase inhibitor than is the previously mentioned recombinant tridegin polypeptide prepared from Escherichia coli.

The present invention therefore relates to a modified tridegin polypeptide which is derived from SEQ ID No. 1 and which possesses one or more modifications.

Within the meaning of this invention, a polypeptide is understood as denoting peptides having more than 15 amino acids (AA) and less than 2000 amino acids, preferably peptides having more than 15 AA and less than 500 AA, in particular peptides having more than 16, 17, 18, 19 or 20 AA and less than 400, 300, 200, 100, 80, 60, 50, 40 or 30 AA.

Within the meaning of this invention, a modification is understood as denoting a change in the wild-type tridegin polypeptide brought about by the replacement of at least one cysteine residue with another amino acid and/or the replacement of at least one of the following amino acids—Lys2, Lys7, His10, Gly12, Leu24, Tyr31, Phe34, Arg39, Ile45, Met48, Asp50, Pro55, Phe58, Asn60, Pro65, Arg66—with another amino acid and/or a deletion of the N and C termini, and/or a covalent linkage to polyethylene glycol.

Within the meaning of this invention, a replacement is understood as denoting the replacement of an amino acid at a particular site in the amino acid sequence of a polypeptide with another amino acid, preferably with one of the other 19 natural amino acids.

Within the meaning of this invention, a deletion is understood as denoting the removal of N- and/or C-terminal regions of the amino acid sequence of the tridegin polypeptide, e.g. the removal of in all more than 5, 10, 15, 20, 25, 30, 35 or even 40 amino acids (with this meaning the sum of the amino acids removed at the N and C termini), with the remaining polypeptide still at least containing the amino acid sequence DDIYGRPVEFPNLPL (SEQ ID No. 92) or DDIYGRPVEFPNLPLK (SEQ ID No. 47).

Surprisingly, the modified tridegin polypeptides exhibited the following advantageous properties when compared with wild-type tridegin polypeptide which is expressed in Escherichia coli.

Whereas the recombinant wild-type tridegin polypeptide which was obtained from Escherichia coli also formed high molecular weight aggregates, the modified tridegin polypeptides in which at least one cysteine residue, preferably from 1 to 4 cysteine residues, particularly preferably 3 or 4 cysteine residues, was/were replaced with another amino acid, preferably a small amino acid, such as valine, alanine, glycine or serine, particularly preferably with alanine or serine, especially with alanine, exhibited a reduction in aggregate formation. This was established in comparative analytical gel filtration runs. The experimental conditions for the gel filtration runs are given in implementation example 4, to which the reader is referred.

In addition, the said modified tridegin polypeptides exhibit a slower formation of the high molecular weight aggregates than does wild-type tridegin during storage at 4° C. This is established in comparative analytical gel filtration runs. The experimental conditions for the gel filtration runs are given in implementation example 4, to which the reader is referred.

Whereas the wild-type tridegin polypeptide which was expressed in Pichia pastoris was cleaved by one or more proteases at its extreme C terminus, it was surprisingly possible to express the variant Arg66Leu completely, and purify it. As compared with the wild-type tridegin polypeptide which was expressed in Pichia pastoris, an inhibitory activity was observed which was essentially unchanged but which was better than that of the wild-type tridegin polypeptide which was expressed in E. coli.

Tridegin polypeptides which have been secreted by their host may possess a higher specific activity than do intracellularly produced tridegin polypeptides, as demonstrated here taking the tridegin obtained from Pichia pastoris as an example, and may consequently be particularly advantageous. For this reason, recombinant tridegin polypeptides which can be obtained by secretion from a host are part of the subject-matter of the invention, in particular when they are obtained by secretion from a host. In the same way, a process for preparing tridegin polypeptides which uses tridegin polypeptides which have been released by their host, in a secretion step, to the outside of the cell, in particular to the medium, is part of the subject-matter of the invention. This can apply to all recombinant methods for preparing tridegin polypeptide which comprise a step of secreting the tridegin polypeptide. Such a secretion step can exist if the tridegin polypeptide crosses a cell membrane in the host. The secretion step can take place during the synthesis of the tridegin polypeptide or else after the polypeptide is already present in the cell.

Secretion of a recombinant polypeptide of the invention, which polypeptide is expressed in a recombinantly manipulable host, can be achieved by using suitable molecular biological methods, e.g. as described in Sambrook et al., “Molecular Cloning: A Laboratory Manual.” Third edition (2001) CSHL Press, to produce a DNA expression vector which comprises a nucleic acid which is under the control of a promoter which is suitable for the expression in the corresponding host and which encodes a polypeptide which comprises what is termed a signal peptide, preferably at its N terminus. Signal peptides can be recognized by the secretion machinery of the cell and can mediate translocation of a protein through a cell membrane. The translocation process is in general mediated by the translocation machinery, which forms a type of channel for specific proteins through the lipid membrane. In general, but not in every case, the signal peptide of a secreted protein is separated off during the translocation through this channel. The mode of functioning, and the components, of the translocation machinery are discussed in Rapoport T. A., et al., Annu. Rev. Biochem. (1996) 65: 271-303, as are common features and differences in the translocation machinery in eukaryotes and prokaryotes. While the host for the expression and secretion of a polypeptide of the invention can be any microbiological host, the host can also be higher eukaryotic cells in culture, such as human cells (e.g. HeLa cells) or insect cells (e.g. insect cells which can be infected with baculovirus so as to achieve ectopic protein expression), as long as the host can be manipulated using recombinant methods and is able to secrete recombinant proteins. The microbial host can be an archaebacterium, a eubacterium or a lower eukaryote, such as a fungus (such as acrasiomycetes, myxomycetes, phycomycetes, ascomycetes, basidomycetes or fungi imperfecti, in particular yeasts such as Pichia pastoris or Saccharomyces cerevisiae), or a protist (such as flagellates, rhizopoda, sporozoa or ciliates, in particular slime molds such as Dictostelium discoideum as well). As a result of being transfected with suitable vectors, cells of higher eukaryotes, such as mammalian cell lines, can also express proteins in the cytoplasm (e.g. pcDNA3.1, Invitrogen Inc.) or express them such that they are secreted (e.g. pSecTag2, Invitrogen Inc.) (see, e.g., “Mammalian Cell Biotechnology: A Practical Approach, by M. Butler (editor), IRL Press, Oxford-New York-Tokyo, page 9, line 23: examples 6 and 7). Suitable host cells for this purpose include CHO cells and HEK293 cells. In particular, the host can be a Gram-negative bacterium, such as Escherichia coli or Serratia marcescens. In these bacteria, secreted, recombinant proteins can be released to the periplasm and these secreted proteins can be isolated without disrupting the host cell itself. Suitable signal peptides for use in Gram-negative bacteria, for example for Escherichia coli, are described in Pines O. and Inouye M., Mol. Biotechnol. (1999) 12: 25-34.

In particular, the host can be a Gram-positive bacterium, such as Bacillus subtilis and related Bacillus species, such as B. amyloliquefaciens or B. licheniformis, since these bacteria are likewise able to release proteins to the culture medium. Suitable signal peptides for use in Gram-positive bacteria, for example for B. subtilis, are described in Tjalsma H., et al., Microbiology and Molecular Biology Reviews, (2000) 64: 515-547.

Preference is also given to using a lower eukaryote as host since recombinant proteins which are secreted by these lower eukaryotes can be released to the medium and it is consequently likewise not always necessary to disrupt the host cell. Suitable signal peptides for use in eukaryotes are described, for example, in Rapoport T. A. et al., Annu. Rev. Biochem. (1996) 65: 271-303. In addition, the reader is referred to the alpha factor signal peptide which is used in example 6 and to Kjeldsen T., Appl. Microbiol. Biotechnol. (2000) 54(3): 277-86 and Brake A. J. Biotechnology (1989) 13:269-80. Without being bound to a particular theory, the passage of the secreted tridegin polypeptide through the translocation machinery, which is partially conserved evolutionarily between bacteria and eukaryotes, appears to provide the polypeptide with a fold, something which is advantageous. In addition, it might be the case that quality control mechanisms which are active in connection with secretion are responsible for ensuring that the secreted tridegin polypeptide is essentially free of incorrectly folded tridegin polypeptide, thereby making it possible for the secreted tridegin polypeptides to have a high specific inhibitory activity. In addition, as a result of the secretion step, the tridegin polypeptide passes from the reducing environment of the cytoplasm into oxidizing cell compartments, thereby facilitating the formation of disulfide bridges.

The modified tridegin polypeptides in which at least one, preferably from one to ten, particularly preferably from one to six, especially from one to three, but in particular only one of the following amino acids—Lys2, Lys7, His10, Gly12, Leu24, Tyr31, Phe34, Arg39, Ile45, Met48, Asp50, Pro55, Phe58, Asn60, Pro65, Arg66—has/have been replaced by another amino acid, preferably a small amino acid such as valine, alanine, glycine or serine, particularly preferably by alanine and glycine, especially by alanine, surprisingly exhibit less antigenicity than the wild-type tridegin, with this surprisingly being in conjunction with an inhibitory activity on human factor XIIIa which is comparable to that of the wild-type tridegin polypeptide, something which was in turn surprising.

In the search for a minimal amino acid sequence which was derived from the wild-type tridegin polypeptide and which inhibited factor XIIIa, it was surprisingly found that polypeptides which at least contained the amino acid sequence DDIYQRXVXFPXLPL, in particular the amino acid sequence DDIYQRPVEFPNLPL or DDIYGRPVEFPNLPLK exhibited an inhibitory effect on human factor XIIIa. Thus, even polypeptides of only 16 amino acids in length which contained the abovementioned amino acid sequence inhibited human factor XIIIa. Variants of the wild-type tridegin polypeptide-derived polypeptides which possessed inhibitory activity and in which in each case one amino acid was replaced with alanine confirmed these results. Furthermore, these experiments make it possible to deduce the residues which are essential for the inhibitory effect.

The consequences of the alanine substitution in the original polypeptide SEQ ID No. 25 (substituted residues in the sequence marked with an X) can be summarized as follows:

Starting sequence (SEQ ID No. 25): PMDDIYQRPVEFPNLPLKPR Substitution without any decrease in activity in the case of the following X amino acids: XXDDIYQRXVXFPXLPLKXX

Substitution with a slight decrease in activity in the case of the following X amino acids: PMXXIYXXPXEXXNXXLXPR

Substitution with a greater decrease in activity in the case of the following X amino acids: PMDDXXQRPVEFPNLPXKPR

It follows from these results that the minimal FXIIIa-inhibiting polypeptide has the following sequence: DDIYQRXVXFPXLPL (SEQ ID No. 89), with the amino acids denoted with an X being able to be, independently of each other, any amino acids which are preferably selected from the natural amino acids, in particular small amino acids such as valine, alanine, glycine or serine, particularly preferably alanine and glycine, especially, however, alanine. One, two or three of the amino acids denoted by X in the above SEQ ID No. 89 sequence can also be the wild-type amino acids for the corresponding site.

Short polypeptides which contain less than 40, preferably less than 30, particularly preferably less than 25, amino acids, and which [lacuna] at least the amino acid sequence DDIYQRXVXFPXLPL (SEQ ID No. 89), with it being possible for the amino acids denoted by X to be, independently of each other, any amino acids, preferably selected from the natural amino acids, in particular small amino acids such as valine, alanine, glycine or serine, particularly preferably alanine and glycine, especially, however, alanine, and with it being possible for one, two or three of the amino acids denoted by X in the above SEQ ID No. 89 sequence also to be the wild-type amino acids for the corresponding site, in particular those which the amino acid sequence DDIYQRPVEFPNLPL or DDIYQRPVEFPNLPLK contain, possess the additional advantage that they have less tendency to aggregate, can be synthesized chemically in large quantities and exhibit less antigenicity than does the wild-type tridegin polypeptide.

Further advantageous, modified tridegin polypeptides are tridegin polypeptides which are linked covalently to polyethylene glycol. The reaction conditions for the modification with PEG are described, for example, in Cohen et al., Biochem. J., 357(3): 795-802 (2001). The polyethylene glycol which is used in the modification reaction should have a molecular weight of from 500 Da to 20 000 Da, preferably between 1000 Da and 10 000 Da, particularly preferably between 2000 Da and 5000 Da. It should be used in a molar ratio of polyethylene glycol:polypeptide according to the invention of between 0.5:1 and 10:1, preferably between 0.8:1 and 4:1, particularly preferably between 1:1 and 2:1. These modified polypeptides have the advantage that, following injection into the blood stream of a mammal, they are less rapidly broken down than is the unmodified polypeptide.

The invention furthermore relates to compounds which contain the above-described polypeptides.

These compounds include, in particular, fusion proteins which have a content of an amino acid sequence which is not derived from the tridegin polypeptide but is, for example, derived from another protein of 5-500, preferably 5-400, 5-300, 5-200, 5-100, 5-50, especially 5-20, amino acids (LaVallie and McCoy, Curr. Opin. Biotechnol. 6(5): 501-506 (1995)), as well as fusion proteins which have a content of an amino acid sequence which is not derived from the tridegin polypeptide but which is derived, for example, from another protein of more than 5, 6, 7, 8, 9, 10, 12, 14, 16, 18, 20, 25, 30 or 50 amino acids and less than 500, 450, 400, 350, 300, 250, 200, 150, 100, 75, 50 or 40 amino acids and all permutations in isolated form thereof. In this connection, the content of an amino acid sequence which is derived from the tridegin polypeptide is preferably less than 50, 45, 40, 35, 30, 25 or 20 amino acids.

Examples of such amino acid sequences which are derived from a foreign protein are prokaryotic peptide and polypeptide sequences which can be derived, for example, from the Escherichia coli galactosidase. It would furthermore also be possible to use viral peptide and polypeptide sequences, for example from the bacteriophage N13, in order, in this way, to generate fusion proteins for the phage-display method known to the skilled person (McCafferty et al., Nature 348(6301): 552-554 (1990)). In addition, it is also possible to use eukaryotic polypeptide sequences, for example from the green fluorescent protein (GFP, described in Prasher et al., Gene 111(2): 229-233 (1992)), in order, in this way, to generate fluorescent fusion proteins which can be detected in vivo. It is also possible to use variants of GFP (Tsien, Annu. Rev. Biochem. 67: 509-544 (1998)) as well as the red fluorescent protein. Furthermore, glucose dehydrogenase and polypeptide fragments thereof can be excluded as fusion partners.

Other preferred examples of peptide and polypeptide sequences for fusion proteins are peptides which facilitate the purification of the above-described fusion proteins, i.e. what are termed tags, and can consequently be used for purifying the polypeptides according to the invention (see Nilsson et al., Protein Expr. Purif. 11(1): 1-16 (1997)). Tags on the poly-peptides according to the invention make it possible, for example, for the polypeptides to be absorbed, with high affinity, on a matrix and to be washed stringently with suitable buffers without eluting the complex between the fusion protein and the matrix to any significant extent, and subsequently for the fusion protein which is bound to the matrix to be eluted selectively. Examples of such tags are a (His)₆ tag, with it already being possible to use five consecutive histidines as a tag for the purification, a Myc tag, a FLAG tag, a chitin-binding tag, the polypeptide gluta-thione transferase (GST) and the polypeptide maltose-binding protein (MBP). The skilled person is familiar with other tags which have an equivalent function.

Other preferred examples of peptide and polypeptide sequences for fusion proteins are peptides and poly-peptides which mediate the secretion of the above-described polypeptides from a host. Examples of such peptide and polypeptide sequences can be found in Pines O. and Inouye M., see above; Rapoport T. A., et al., see above, and Tjalsma H., et al., see above.

The invention furthermore relates to a process for preparing the above-described polypeptides. Thus, the polypeptides according to the invention can be prepared using recombinant methods or methods of peptide chemistry.

A recombinant method for preparing one of said poly-peptides consists, for example, in cloning a nucleic acid, which encodes one of the described polypeptides, into prokaryotic or eukaryotic expression vectors in a suitable manner (Sambrook et al., “Molecular cloning: a laboratory manual” Second edition, Cold Spring Harbor Laboratory Press (1989); Sambrook et al., “Molecular cloning: a laboratory manual” Third edition, Cold Spring Harbor Laboratory Press (2001)). Such expression vectors comprise at least one promoter, at least one translation initiation signal, at least one nucleic acid sequence which encodes one of the polypeptides according to the invention and a translation termination signal, in the case of prokaryotic expression vectors, and additionally a transcription termination signal and also a polyadenylation signal in the case of eukaryotic expression vectors. A nucleic acid which encodes one of the polypeptides according to the invention can, for example, be part of a vector, such as a plasmid, a phageimid, a cosmid, a BAC or a YAC, in particular part of a prokaryotic or eukaryotic expression vector (Sambrook et al., “Molecular Cloning: A Laboratory Manual”, third edition, “Cold Spring Harbor Laboratory Press” (2001); plasmids described in 1.3-1.29, phagimids described in 3.42-3.52, cosmids described in 4.1-4.10 and eukaryotic expression vectors in 17.83-17.111). Further examples of prokaryotic expression vectors are, e.g., expression vectors based on promoters which are recognized by T7 RNA polymerase, as described in U.S. Pat. No. 4,952,496, which are suitable for expression in Escherichia coli, the expression vectors which were described in Le Grice S. F. J. in Methods in Enzymol. (1990) vol. 185, pages 201-214, which are suitable for expression in, e.g. Bacillus subtilis, or those described by Nagarajan V. in Methods in Enzymol. (1990) vol. 185, pages 214-223, which are suitable for secretion in B. subtilis, while examples of eukaryotic expression vectors are, e.g. the vectors p426Met25 or p526GAL1 (Mummberg et al. (1994) Nucl. Acids Res., 22, 5767-5768) or the vectors described in Methods in Enzymol. (1990) vol. 185, pages 297-329, described by Mylin L. M., et al. (297-308), Price V. L., et al., (308-319) and Etcheverry T. (319-329), which are suitable for expression in Saccharomyces cerevisiae, or vectors as described in Methods in Enzymol. (1990) vol. 185, pages 408-440, described by Brake A. J. (408-421) and Hitzeman R. A., et al. (421-440), which are suitable for secretion in S. cerevisiae, the vectors as described, for example, in Cregg J. M. et al., Mol. Biotechnol. (2000) 16(1): 23-52 or in “Pichia Protocols” D. R. Higgins and J. M. Cregg (ed.) Humana Press, Totowa, N.J., which are suitable for expression in Pichia Pastoris, the vectors described in Gellissen G., Appl. Microbial. Biotechnol. (2000) 54(6): 741-50, which are suitable for expression in other yeasts, e.g. in Hansenula polymorpha, e.g. Baculovirus vectors as disclosed in EP-B1-0 127 839 or EP-B1-0 549 721, which are suitable for expression in insect cells, and, e.g., the vectors Rc/CMV and Rc/RSV or SV40 vectors, or the vectors described by Kaufman R. J. in Methods in Enzymol. (1990) vol. 185, pages 487-512, which are suitable for expression in mammalian cells, with all these vectors being generally available (for further relevant expression systems, see also Andersen D. C. and Krummen L., Curr. Opin. Biotechnol. (2002) 13(2):117-23).

The skilled person is familiar with the molecular biological methods for preparing these expression vectors as well as the methods for introducing the expression vectors into the host cells and also the conditions for culturing the transformed host cells and, finally, the conditions for inducing the expression of the desired polypeptide according to the invention in the host cells (see also Sambrook et al., see above). Examples of the recombinant preparation of polypeptides according to the invention are given in implementation examples 1, 2 and 4.

However, the above-described polypeptides can also be prepared, as in implementation example 3, by means of a method involving peptide chemistry, that is, for example, using the well-known solid phase synthesis as described in Merrifield, J. Am. Che. Soc. 85: 2149 (1962). Techniques for synthesizing and purifying peptides are also described, for example, on pages 27-62 in Stewart and Young, “Solid Phase Peptide Synthesis” (Freeman, San Francisco, 1969), as well as in U.S. Pat. No. 4,269,827.

The invention also relates to the use of one of the abovementioned polypeptides as an inhibitor of trans-glutaminases, in particular of factor XIIIa, especially of human factor XIIIa. The abovementioned polypeptides have the property of inhibiting the factor XIIIa-catalyzed release of ammonium ions which, in the factor XIIIa-catalyzed reaction, are released from a specific peptide substrate using glycine ethyl ester, as, for example, in the Behrichrom® assay (from Dade Behring GmbH, Marburg). This inhibitory effect of the above-mentioned polypeptides can be detected, for example, in the Behrichrom® assay, as described in implementation examples 1 to 4.

In addition, the polypeptides according to the invention are able to inhibit the mammalian proteins which are homologous to human factor XIIIa, for example the Rattus norvegicus protein which is homologous with human factor XIII and in which 617 out of 732 amino acids are identical (84%) and 689 out of 732 amino acids are related (93%).

Aside from their inhibitory effect on factor XIIIa, the abovementioned polypeptides also inhibit other trans-glutaminases, e.g. the transglutaminase 1 which is expressed in the keratinocytes, the transglutaminase 3 which is involved in the formation of the epidermis, the transglutaminase 4 which, in the vas deferens, is involved in the crosslinking of proteins and the conjugation of polyamines, as well as the trans-glutaminase which is involved in the keratinization of keratinocytes, and consequently all six of the human proteome transglutaminases which have thus far been described.

Another embodiment of the invention consists in using a polypeptide according to the invention for preventing and treating thromboses. Because the polypeptides according to the invention inhibit factor XIIIa, they also inhibit the formation of fibrin polymer cross-linkages. This thereby inhibits the formation of “hard” blood thrombi, which are resistant to being broken down by fibrinolytic enzymes.

For example, the polypeptides according to the invention enabled human blood thrombi to be lyzed more rapidly and also inhibited the onset of blood coagulation, as described in implementation example 5. They are consequently suitable for preventing and treating thromboses.

The invention furthermore relates to a pharmaceutical which comprises a polypeptide according to the invention and at least one galenic adjuvant. While the poly-peptides according to the invention are potent trans-glutaminase inhibitors, they exhibit only a slight degree of toxicity and can therefore be particularly readily used for producing pharmaceuticals.

According to the invention, the term “galenic adjuvant” denotes any inert, nontoxic, solid or liquid filler, diluent or packaging material, as long as it does not react with the polypeptide according to the invention or the patient in an unacceptably disadvantageous manner. Examples of liquid galenic adjuvants are sterile water, physiological sodium chloride solution, sugar solutions, ethanol and/or oils. Galenic adjuvants for producing tablets and capsules can, for example, comprise binders and fillers.

The invention also relates to a combination preparation which comprises a polypeptide according to the invention as well as at least one pharmaceutical active compound.

A preferred embodiment of the invention consists of a combination preparation which comprises at least one of the peptides according to the invention as well as an additional active compound in the form of an anti-coagulant. Anticoagulants either promote the lysis of blood thrombi or inhibit the formation of blood thrombi. Examples are thrombolytic active compounds, that is active compounds which promote the breakdown of active thrombin or prothrombin, fibrinolytic active compounds, that is active compounds which promote the breakdown of polymeric fibrin, or fibrinogenolytic active compounds, that is active compounds which promote the breakdown of fibrinogen. Preference is given to anticoagulants which are activators of plasmin or plasminogen or inhibitors of thrombin and factor Xa, or inhibitors of blood platelet aggregation.

Particularly preferred anticoagulants which can be used jointly with the peptides according to the invention in combination preparations are acetylsalicylic acid, heparin, low molecular weight heparin, heparinoid, hirudin, bivalirudin, melagatran, abciximab, eptifibabide, tissue plasminogen activator (tPA), streptokinase, staphylokinase, urokinase, eminase, hementin and/or plasmin.

Acetylsalicylic acid acts, inter alia, as an inhibitor of blood platelet aggregation. Heparin is an endogenous polyanionic polysaccharide which has a molecular weight of from 6000 Da to 30 000 Da and increases the activity of the endogenous antithrombin III. Low molecular weight heparin is obtained by the limited breakdown of heparin and has a molecular weight of from 4000 Da to 6000 Da. Hirudin is described, for example, in EP 0347376 and EP 0501821. “Hirudin” is used to designate a family of homologous polypeptides which are derived from leeches and which inhibit thrombin and blood coagulation. Bivalirudin is a thrombin-inhibiting peptide (Kelly et al., Proc. Natl. Acad. Sci. USA, 89, 6040-6044 (1992)). Melagatran is a thrombin-inhibiting peptide mimetic (Thromb Haemost 79(1): 110-118 (1998)). Abciximab is an antibody and eptifibabide is a peptide; both bind GP IIb/IIIb, i.e. platelet glycoprotein IIb/IIIb, and inhibit blood platelet aggregation. Hementin is described, for example, in WO 91/15576, is found in a variety of leeches and breaks down fibrinogen and thereby prevents blood coagulation. In the same way, plasmin or eminase lead to the breakdown of fibrin while streptokinase, urokinase, staphylo-kinase and tissue plasminogen activator (tPA) activate plasminogen and lead to fibrin breakdown by generating active plasmin.

A particular advantage of combining the polypeptides according to the invention with the anticoagulants and, where appropriate, an additional pharmaceutical active compound is that blood thrombi are dissolved more rapidly, in a synergic manner, by the combination of active compounds than they are by one active compound on its own. In particular, the combination with fibrinolytic agents such as urokinase and tissue plasminogen activator (tPA) brought about a decrease in stability, and more rapid dissolution, of blood thrombi, as described in detail in implementation example 5.

The invention will now be further clarified below with the aid of the figures and examples without restricting the invention to them.

### DESCRIPTION OF THE FIGURES AND SEQUENCES

FIG. 1: Map of the expression plasmid pET22b-14 (A) and specification of the tridegin poly-peptide-encoding sequence (B). The bases are numbered in accordance with the plasmid map.

FIG. 2: Purification of recombinant wild-type tridegin polypeptide, as examined by means of SDS-PAGE (10% gel).

Lane 1 is total E. coli lysate before loading onto the Ni-NTA column.

Lanes 2-7 are fractions of the elution with imidazole. All the samples were treated with SDS sample buffer and incubated at 95° C. for 5 min.

FIG. 3: Inhibitory effect of the recombinant, purified tridegin polypeptide on factor XIIIa in a Berichom® assay.

Cerulenin (from Calbiochem) was used as control substance.

FIG. 4: Schematic representation of a thrombelastogram.

The parameters which were relevant for the described experiments are CT (clotting time), MCF (maximum clot firmness) and LT (lysis time).

FIG. 5: Thrombelastograms of whole citrate blood in the absence (A, C and E) and presence (B, D and F) of recombinant tridegin.

All the assay samples contain whole citrate blood (300 μl), Ca²⁺ (20 μl of Starteg reagent) and thromboplastin phospholipid (10 μl of Integ reagent).

B: +Transglutaminase inhibitor having SEQ ID No. 1 (10 NM)

C: +Urokinase (25 U)

D: +Transglutaminase inhibitor having SEQ ID No. 1 (10 μM) and urokinase (25 U)

E: +tPA (40.5 ng)

F: +Transglutaminase inhibitor having SEQ ID No. 1 (10 μM) and tPA (40.5 ng)

FIG. 6: Thrombelastograms of whole citrate blood in the absence (A, C and E) and presence (B, D and F) of SEQ ID No. 25.

All the assay samples contain whole citrate blood (300 μl), Ca²⁺ (20 μl of Starteg reagent) and thromboplastin phospholipid (10 μl of Integ reagent).

B: +Transglutaminase inhibitor having SEQ ID No. 25 (20 μM)

C: +Urokinase (25 U)+ inactive control peptide (acetyl-adhesin (1025-1044) amide from Bachem) (20 μM)

D: +Transglutaminase inhibitor having SEQ ID No. 25 (20 μM) and urokinase (25 U)

E: +tPA (40.5 ng)+inactive control peptide (20 μM)

F: +Transglutaminase inhibitor having SEQ ID No. 25 (20 μM) and tPA (40.5 ng)

FIG. 7: Inhibitory effect of the tridegin-derived peptides and their variants on factor XIIIa in a Berchrom® assay (A-D). Recombinant, purified E. coli tridegin, at the given concentrations, and peptide 25 (SEQ ID No. 25; final concentration in the assay ˜7.27 μM) were used as controls. The sequences of the peptides employed are listed under (E). The error bars relate to the standard error (n=3).

| | | |
|---|---|---|
| A1: | PMDDIYQRPVEFPNLPLKPR | SEQ ID No. 25 |
| A2: | PMDDIYQRPVEFPNLPLKPA | SEQ ID No. 67 |
| A3: | PMDDIYQRPVEFPNLPLKAR | SEQ ID No. 68 |
| A4: | PMDDIYQRPVEFPNLPLAPR | SEQ ID No. 69 |
| A5: | PMDDIYQRPVEFPNLPAKPR | SEQ ID No. 70 |
| A6: | PMDDIYQRPVEFPNLALKPR | SEQ ID No. 71 |
| A7: | PMDDIYQRPVEFPNAPLKPR | SEQ ID No. 72 |
| A8: | PMDDIYQRPVEFPALPLKPR | SEQ ID No. 73 |
| A9: | PMDDIYQRPVEFANLPLKPR | SEQ ID No. 74 |
| A10: | PMDDIYQRPVEAPNLPLKPR | SEQ ID No. 75 |
| A11: | PMDDIYQRPVAFPNLPLKPR | SEQ ID No. 76 |
| A12: | PMDDIYQRPAEFPNLPLKPR | SEQ ID No. 77 |
| B1: | PMDDIYQRAVEFPNLPLKPR | SEQ ID No. 78 |
| B2: | PMDDIYQAPVEFPNLPLKPR | SEQ ID No. 79 |
| B3: | PMDDIYARPVEFPNLPLKPR | SEQ ID No. 80 |
| B4: | PMDDIAQRPVEFPNLPLKPR | SEQ ID No. 81 |
| B5: | PMDDAYQRPVEFPNLPLKPR | SEQ ID No. 82 |
| B6: | PMDAIYQRPVEFPNLPLKPR | SEQ ID No. 83 |
| B7: | PMADIYQRPVEFPNLPLKPR | SEQ ID No. 84 |
| B8: | PADDIYQRPVEFPNLPLKPR | SEQ ID No. 85 |
| B9: | AMDDIYQRPVEFPNLPLKPR | SEQ ID No. 86 |
| B10: | MDDIYQRPVEFPNLPLKPR | SEQ ID No. 48 |
| B11: | DDIYQRPVEFPNLPLKPR | SEQ ID No. 49 |
| B12: | DIYQRPVEFPNLPLKPR | SEQ ID No. 50 |
| C1: | IYQRPVEFPNLPLKPR | SEQ ID No. 51 |
| C2: | YQRPVEFPNLPLKPR | SEQ ID No. 52 |
| C3: | QRPVEFPNLPLKPR | SEQ ID No. 53 |
| C4: | RPVEFPNLPLKPR | SEQ ID No. 54 |
| C5: | PVEFPNLPLKPR | SEQ ID No. 55 |
| C6: | VEFPNLPLKPR | SEQ ID No. 56 |
| C7: | EFPNLPLKPR | SEQ ID No. 57 |
| C8: | PMDDIYQRPVEFPNLPLKP | SEQ ID No. 58 |
| C9: | PMDDIYQRPVEFPNLPLK | SEQ ID No. 59 |
| C10: | PMDDIYQRPVEFPNLPL | SEQ ID No. 60 |
| C12: | PMDDIYQRPVEFPNLP | SEQ ID No. 61 |
| D1: | PMDDIYQRPVEFPNL | SEQ ID NO. 62 |
| D2: | PMDDIYQRPVEFPN | SEQ ID NO. 63 |
| D3: | PMDDIYQRPVEFP | SEQ ID No. 64 |
| D4: | PMDDIYQRPVE | SEQ ID No. 65 |
| D5: | PMDDIYQRPV | SEQ ID No. 66 |

FIG. 8: Map of the expression plasmid trideginpPICZαA (A) and specification of the tridegin poly-peptide-encoding sequence (B). The bases are numbered in accordance with the plasmid map.

FIG. 9: Inhibitory effect of the recombinant, purified tridegin polypeptide (A), or of the Pichia pastoris (KM71H)-derived variant trideginR66L (B) on factor XIIIa in a Berichrom® assay.

FIG. 10: Thrombelastograms of whole citrate blood in the absence (A, D and G) and presence (B, C, E, F, H and I) of SEQ ID No. 87 or SEQ ID No. 25 (both 25 μM). All the assay samples contain whole citrate blood (300 μl), Ca²⁺ (20 μl of Starteg reagent) and thromboplastin phospholipid (10 μl of Integ reagent).

B: +Transglutaminase inhibitor (TI) having SEQ ID No. 87 (25 μM)

C: +TI having SEQ ID No. 25 (25 μM)

D: +Urokinase (25 U)

E: +TI having SEQ ID No. 87 (25 μM)+urokinase (25 U)

F: +TI having SEQ ID No. 25 (25 μM)+uro-kinase (25 U)

G: +tPA (40.5 ng)

H: +TI having SEQ ID No. 87 (25 μM)+tPA (40.5 ng)

I: +TI having SEQ ID No. 25 (25 μM)+tPA (40.5 ng)

FIG. 11: Thrombelastograms of whole citrate blood in the absence (A, C and E) and presence (B, D and F) of recombinant, purified Pichia pastoris-derived trideginR66L (encoded by SEQ ID No. 91). All the assay samples contain whole citrate blood (300 μl), Ca²⁺ (20 μl of Starteg reagent) and thromboplastin phospholipid (10 μl of Integ reagent).

B: +Transglutaminase inhibitor (TI) having SEQ ID No. 91 (5 μM)

C: +Urokinase (25 U)

D: +TI having SEQ ID No. 91 (5 μM)+uro-kinase (25 U)

E: +tPA (40.5 ng)

F: +TI having SEQ ID No. 91 (5 μM)+tPA (40.5 ng)

```
SEQ ID No. 1:
Met Lys Leu Leu Pro Cys Lys Glu Trp His Gln Gly Ile Pro Asn Pro
1 5 10 15
Arg Cys Trp Cys Gly Ala Asp Leu Glu Cys Ala Gln Asp Gln Try Cys
                        20 25 30
Ala Phe Ile Pro Gln Cys Arg Pro Arg Ser Glu Leu Ile Lys Pro Met
                35 40 45
Asp Asp Ile Tyr Gln Arg Pro Val Glu Phe Pro Asn Leu Pro Leu Lys
        50 55 60
Pro Arg Glu Glu
65
```

SEQ ID No. 1 shows the wild-type tridegin polypeptide.
SEQ ID No. 2 to SEQ ID No. 26 in each case show 20 amino acid-long peptides from SEQ ID No. 1.
SEQ ID No. 27 to SEQ ID No. 46 show oligonucleotides used for mutagenizing the tridegin polypeptide.
SEQ ID No. 47 shows a 16 amino acid-long peptide from
SEQ ID No. 1 while SEQ ID No. 92 shows a 15 amino acid-long peptide from SEQ ID No. 1.
SEQ ID No. 48 to SEQ ID No. 88 show truncated peptides and peptide variants.
SEQ ID No. 89 shows a 15 amino acid-long peptide.
SEQ ID No. 90 and SEQ ID No. 91 show coding DNA sequences used for the expression in Pichia pastoris.

### Implementation Examples

### Example 1

### Expressing and Purifying Recombinant Tridegin Polypeptide (SEQ ID NO: 1) from Escherichia Coli

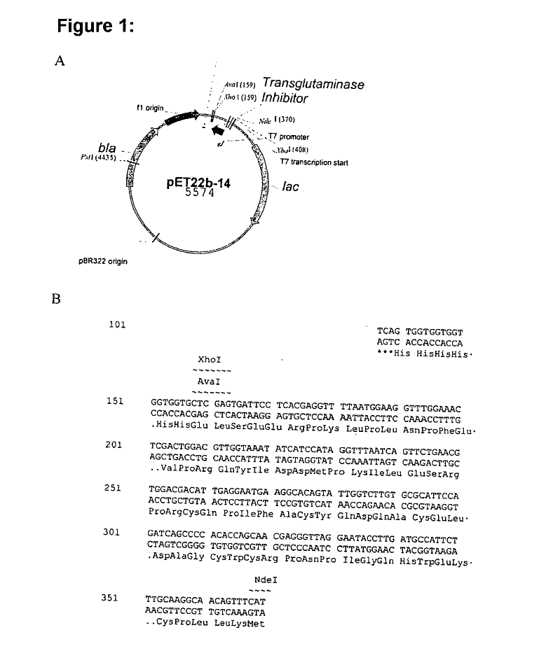

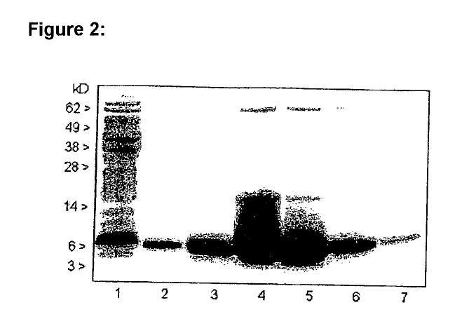

The expression plasmid pET22b-14, which contains the sequence encoding the recombinant tridegin polypeptide, is depicted in FIG. 1. Current methods were used to transfer the plasmid into the Escherichia coli expression strain Origami® B (DE3) (from Novagen, order No. 70837), after which the strain was cultured in liquid LB medium containing ampicillin (100 μg/ml), kanamycin and tetracycline (in each case 5 μg/ml). The expression strain BL21 (DE3) (from Novagen) gave similar results and can also be used for expressing the modified tridegin polypeptides. The main culture was shaken at 37° C. and 220 rpm until an OD600 of 0.7 had been reached. At a cell density of 0.7-0.9 OD600/ml, the culture was treated with 2 mM IPTG/ml, in order to induce the gene expression, and then shaken at 37° C. and 200-240 rpm for a further 4 h. The cells were harvested by subsequent centrifugation (15 min, 5825×g). The cell sediment was resuspended in 10× BugBuster protein extraction reagent (from Novagen), which had been diluted 1:10 with highly pure water and, for the purposes of disruption, incubated at 4° C. for 10-20 min, while being shaken, with benzonase (from Novagen) and protease inhibitor cocktail (Complete® without EDTA) from Roche Diagnostics GmbH). The super-natant was obtained by subsequently centrifuging at 16 000×g and 4° C. for 20 min, and then treated with an equal volume of lysis buffer (50 mM NaH₂PO₄, pH 8.0, 300 mM NaCl, 10 mM imidazole). The resulting protein suspension was stored at 4° C. overnight. For the purification, 3 ml of nickel NTA agarose (from Quiagen) were packed into an empty column and equilibrated with 5 column volumes of lysis buffer. The protein suspension was loaded onto the column (without pumping, flow as a result of gravity) and then washed (10 column volumes) with washing buffer (50 mM NaH₂PO₄ pH 8.0, 300 mM NaCl, 20 mM imidazole). The column was eluted with elution buffer (2-3 column volumes) (50 mM NaH₂PO₄, 300 mM NaCl, 250 mM imidazole, pH 8). Fractions were collected and examined by SDS-PAGE for the presence of trans-glutaminase inhibitor (see FIG. 2). The yield was 20 mg of recombinant tridegin polypeptide per liter of expression culture, with the purity being >90%. The fractions which contained the recombinant tridegin polypeptide were combined and dialyzed extensively against 50 mM NaH₂PO₄, pH 8.0, 300 mM NaCl (2× against 21). The inhibitory activity of the purified protein on factor XIIIa was then tested. The Berichrom® assay (from Dade Behring) was used as the test method.

### Implementing the Behrichrom® Assay:

The assay is based on factor XIII being activated to form factor XIIIa by thrombin which is present in the reagents. Factor XIIIa links a specific peptide substrate to glycine ethyl ester with ammonium ions being released. The latter are determined in an enzyme reaction which proceeds in parallel. The extinction at 340 nm was used to measure the decrease in NADH. For the measurement, peptides were present in 50% acetonitrile in a stock concentration of 5 mM. The NADH and detection reagents which were supplied by the manufacturer were dissolved in 3 ml of water, with the activator reagent subsequently being dissolved in 3 ml of NADH reagent. For use, the activator and detection reagents were mixed in a ratio of 1:1. In order to carry out the measurement in microtiter plate format, 100 μl of sample (inhibitor or control buffer), 25 μl of factor XIII (10 U/ml) and 150 μl of working reagent were mixed. The measurement was carried out continuously for 20 min at 340 nm, and at 37° C., in a microtiter plate photometer. For the evaluation, the differences in the values measured after 16 and 20 min were compared.

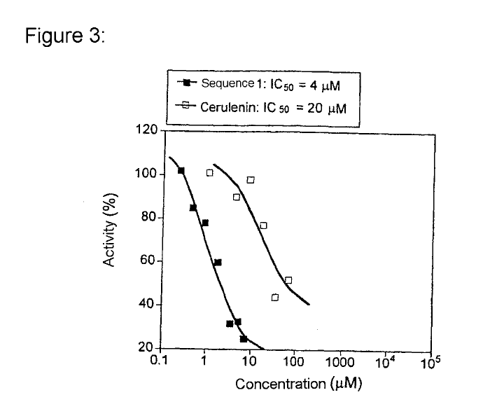

The measurement gave an IC₅O of 2-4 μM (see FIG. 3) for the purified transglutaminase inhibitor.

### Example 2

### Expressing and Purifying Modified Tridegins from Escherichia Coli

Modified tridegins were produced by site-directed mutagenesis of the expression plasmid encoding the wild-type tridegin polypeptide. The mutagenesis was carried out by PCR using the QuikChange reagents (from Strategene) in accordance with the manufacturer's instructions. The oligonucleotides SEQ ID No. 27 to SEQ ID No. 40 and the respective reverse-complementary sequences were used for the mutagenesis.

The DNA sequences of the resulting mutants were checked by sequencing. Current methods were used to transfer the respective coding sequence, in plasmid pET22b, into the Escherichia coli expression strain Origami® B(DE3) (from Novagen), with the strain then being cultured in liquid LB medium containing ampicillin, kanamycin and tetracycline as already described above. Additional, spontaneously arising double mutants which were found were also expressed and purified. Expression, purifi-cation and determination of activity were carried out as described in example 1. The activities shown in table 2 below were measured for the individual modified tridegins. The modified tridegins were named in accordance with the XnY scheme.

In this scheme, X denotes the amino acid which was changed by mutagenesis, while n defines the position of this amino acid in the polypeptide chain and Y denotes the amino acid which is present after the mutagenesis.

TABLE 2
Inhibitory effect of modified tridegins on factor
XIIIa in a Berichrom ® assay

| | Relative inhibitory |
|---|---|
| Variant | effect (%) |
| Wild-type polypeptide | 100 |
| K02A | 113 |
| K07A | 90 |
| H10A | 87 |
| G12A | 97 |
| L24A | 92 |
| Y31A | 69 |
| F34A | 96 |
| R39A | 49 |
| I45A | 115 |
| M48A | 91 |
| D50A | 53 |
| D50A, P55L | 84 |
| F58A | 60 |
| N60A | 68 |
| P65A | 51 |

The following oligonucleotides were used to produce the abovementioned variants:

| | SEQ | |
|---|---|---|
| Oligonucleotide | ID No. | Variant |
| 5′-ccttgatgccattctttgcaaggcaacagtgccatatgtatatctccttc | 33 | K02A |
| 5′-catatgaaactgttgccttgcgcagaatggcatcaaggtattcctaaccc | 34 | K07A |
| 5′-cgagggttaggaataccttgagcccattctttgcaaggcaacag | 31 | H10A |
| 5′-cttgcaaagaatggcatcaagctattcctaaccctcgttgctggtg | 30 | G12A |
| 5′-gtattggtcttgtgcgcattccgcatcagccccacaccag | 35 | L24A |
| 5′-gaggaatgaaggcacaagcttggtcttgtgcgcattccagatc | 40 | Y31A |
| 5′-gtggacgacattgaggaatggcggcacagtattggtcttgtgc | 28 | F34A |
| 5′-ggtttaatcagttctgaacgtggagcacattgaggaatgaaggcacagtattg | 39 | R39A |
| 5′-ttggtaaatatcatccataggtttagccagttctgaacgtggacgacattgagg | 32 | 145A |
| 5′-cgactggacgttggtaaatatcatccgcaggtttaatcagttctgaacgtggacg | 36 | M48A |
| 5′-ctcgactggacgttggtaaatagcatccataggtttaatcagttctgaacgtgg | 27 | D50A |
| 5′-cgaggttttaatggaaggtttggagcctcgactggacgttggtaaatatcatcc | 29 | F58A |
| 5′-cctcacgaggttttaatggaagggctggaaactcgactggacg | 37 | N60A |
| 5′-gtggtgctcgagtgattcctcacgagcttttaatggaaggtttgg | 38 | P65A |

The relative inhibitory effect (%) was determined at a final variant concentration of 5.45 μM. The inhibitory effect of the recombinant tridegin polypeptide was normalized to 100% (at 5.45 μM).

### Example 3

### Inhibitory Effect of Fragments of the Tridegin Polypeptide

A current method of peptide synthesis (from Pepscan, Lelystad, NL) was used to chemically synthesize 25 peptides of 20 amino acids in length on the basis of the recombinant tridegin polypeptide. The peptides carry an acetyl group N-terminally and correspondingly carry an amide group C-terminally. The sequences were selected such that they
a) cover the entire sequence 1, and
b) overlap by in each case 18 amino acid residues (see table 3, sequences 2-26).

The above-described Berichrom® assay was used to determine the relative inhibitory effect (%) at a final concentration of the peptides of 7.27 μM. The inhibitory effect of the recombinant tridegin polypeptide, at a final concentration of 7.27 μM, was normalized to 100%.

TABLE 3

Inhibitory effect of peptides of the recombi-
nant tridegin on factor XIIIa in a Berichrom ®
assay

| | |
|---|---|
| | Relative inhibitory |
| Sequence (Sequence No.) | effect (%) |

| | |
|---|---|
| MKLLPCKEWHQGIPNPRCWC | 0.00 |
| (SEQ ID NO. 2) | |
| LLPCKEWHQGIPNPRCWCGA | 5.88 |
| (SEQ ID NO. 3) | |
| PCKEWHQGIPNPRCWCGADL | 9.80 |
| (SEQ ID NO. 4) | |
| KEWHQGIPNPRCWCGADLEC | 1.96 |
| (SEQ ID NO. 5) | |
| WHQGIPNPRCWCGADLECAQ | 9.80 |
| (SEQ ID NO. 6) | |
| QGIPNPRCWCGADLECAQDQ | 13.73 |
| (SEQ ID NO. 7) | |
| IPNPRCWCGADLECAQDQYC | 13.73 |
| (SEQ ID NO. 8) | |
| NPRCWCGADLECAQDQYCAF | 0.00 |
| (SEQ ID NO. 9) | |
| RCWCGADLECAQDQYCAFIP | 5.88 |
| (SEQ ID NO. 10) | |
| WCGADLECAQDQYCAFEPQC | 0.36 |
| (SEQ ID NO. 11) | |
| GADLECAQDQYCAFIPQCRP | 2.49 |
| (SEQ ID NO. 12) | |
| DLECAQDQYCAFIPQCRPRS | 7.96 |
| (SEQ ID NO. 13) | |
| ECAQDQYCAFIPQCRPRSEL | 6.30 |
| (SEQ ID NO. 14) | |
| AQDQYCAFIPQCRPRSELIK | 3.32 |
| (SEQ ID NO. 15) | |
| DQYCAFIPQCRPRSELIKPM | 14.76 |
| (SEQ ID NO. 16) | |
| YCAFEPQCRPRSELIKPMDD | 12.27 |
| (SEQ ID NO. 17) | |
| AFIPQCRPRSELIKPMDDIY | 13.27 |
| (SEQ ID NO. 18) | |
| IPQCRPRSELIKPMDDIYQR | 0.0 |
| (SEQ ID NO. 19) | |
| QCRPRSELIKPMDDIYQRPV | 11.11 |
| (SEQ ID NO. 20) | |
| RPRSELIKPMDDIYQRPVEF | 19.40 |
| (SEQ ID NO. 21) | |
| RSELIKPMDDIYQRPVEFPN | 8.64 |
| (SEQ ID NO. 22) | |
| ELIKPMDDIYQRPVEFPNLP | 24.01 |
| (SEQ ID NO. 23) | |
| IKPMDDIYQRPVEFPNLPLK | 43.52 |
| (SEQ ID NO. 24) | |
| PMDDIYQRPVEFPNLPLKPR | 42.14 |
| (SEQ ID NO. 25) | |
| DDIYQRPVEFPNLPLKPREE | 35.58 |
| (SEQ ID NO. 26) | |

The Berichrom® assay was used to once again separately measure the inhibitory effects of the three C-terminal peptides (SEQ ID NOs:24, 26 and 26) on factor XIIIa after the peptides had been purified by HPLC. The following IC₅₀ values were measured:
SEQ ID No. 24: IC₅₀: 7 μM
SEQ ID No. 25: IC₅₀: 4 M
SEQ ID No. 26: IC₅₀: 5 μM

In order to determine the minimal length, current methods were used to synthesize 20 peptides (acetylated and amidated) which were truncated by in each case one amino acid either from the C terminus or from the N terminus.

| | | |
|---|---|---|
| B10 | MDDIYQRPVEFPNLPLKPR | SEQ ID No. 48 |
| B11 | DDIYQRPVEFPNLPLKPR | SEQ ID No. 49 |
| B12 | DIYQRPVEFPNLPLKPR | SEQ ID No. 50 |
| C1 | IYQRPVEFPNLPLKPR | SEQ ID No. 51 |
| C2 | YQRPVEFPNLPLKPR | SEQ ID No. 52 |
| C3 | QRPVEFPNLPLKPR | SEQ ID No. 53 |
| C4 | RPVEFPNLPLKPR | SEQ ID No. 54 |
| C5 | PVEFPNLPLKPR | SEQ ID No. 55 |
| C6 | VEFPNLPLKPR | SEQ ID No. 56 |
| C7 | EFPNLPLKPR | SEQ ID No. 57 |
| C8 | PMDDIYQRPVEFPNLPLKP | SEQ ID No. 58 |
| C9 | PMDDIYQRPVEFPNLPLK | SEQ ID No. 59 |
| C10 | PMDDIYQRPVEFPNLPL | SEQ ID No. 60 |
| C12 | PMDDIYQRPVEFPNLP | SEQ ID No. 61 |
| D1 | PMDDIYQRPVEFPNL | SEQ ID No. 62 |
| D2 | PMDDIYQRPVEFPN | SEQ ID No. 63 |
| D3 | PMDDIYQRPVEFP | SEQ ID No. 64 |
| D4 | PMDDIYQRPVE | SEQ ID No. 65 |
| D5 | PMDDIYQRPV | SEQ ID No. 66 |

In order to identify the most important residues, a further 20 peptides were synthesized in which in each case one amino acid was replaced with alanine.

| | | |
|---|---|---|
| A1 | PMDDIYQRPVEFPNLPLKPR | SEQ ID No. 25 |
| A2 | PMDDIYQRPVEFPNLPLKPA | SEQ ID No. 67 |
| A3 | PMDDIYQRPVEFPNLPLKAR | SEQ ID No. 68 |
| A4 | PMDDIYQRPVEFPNLPLAPR | SEQ ID No. 69 |
| A5 | PMDDIYQRPVEFPNLPAKPR | SEQ ID No. 70 |
| A6 | PMDDIYQRPVEFPNLALKPR | SEQ ID No. 71 |
| A7 | PMDDIYQRPVEFPNAPLKPR | SEQ lb No. 72 |
| A8 | PMDDIYQRPVEFPALPLKPR | SEQ ID No. 73 |
| A9 | PMDDIYQRPVEFANLPLKPR | SEQ ID No. 74 |
| A10 | PMDDIYQRPVEAPNLPLKPR | SEQ ID No. 75 |
| A11 | PMDDIYQRPVAFPNLPLKPR | SEQ ID No. 76 |
| A12 | PMDDIYQRPAEFPNLPLKPR | SEQ ID No. 77 |
| B1 | PMDDIYQRAVEFPNLPLKPR | SEQ ID No. 78 |
| B2 | PMDDIYQAPVEFPNLPLKPR | SEQ ID No. 79 |
| B3 | PMDDIYARPVEFPNLPLKPR | SEQ ID No. 80 |
| B4 | PMDDIAQRPVEFPNLPLKPR | SEQ ID No. 81 |
| B5 | PMDDAYQRPVEFPNLPLKPR | SEQ ID No. 82 |
| B6 | PMDAIYQRPVEFPNLPLKPR | SEQ ID No. 83 |
| B7 | PMADIYQRPVEFPNLPLKPR | SEQ ID No. 84 |
| B8 | PADDIYQRPVEFPNLPLKPR | SEQ ID No. 85 |
| B9 | AMDDIYQRPVEFPNLPLKPR | SEQ ID No. 86 |

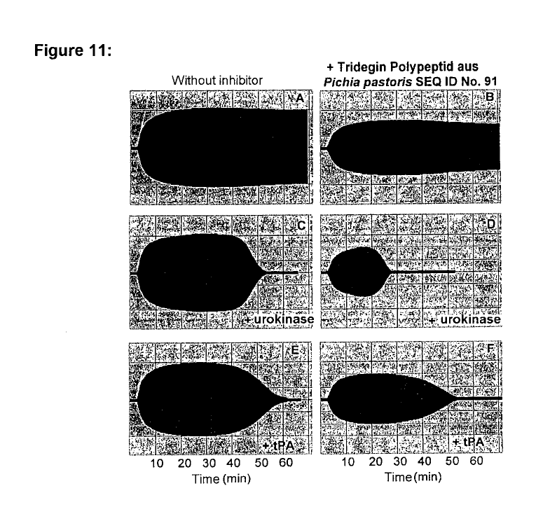

The inhibitory activity of these unpurified peptides (final concentration in the assay ˜7.27 μM) was investigated in the above-described Berichrom assay. The measured values shown in FIG. 7 were obtained.

In order to check the results still further, the two sequences MDDIYQRPVEFPNLPL (SEQ ID No. 87) (16mer) and DDIYQRPVEFPNLP (SEQ ID No. 88) (14mer) were synthesized and purified. The values for the starting sequence (SEQ ID No. 25) were measured for comparison using purified peptide. This then made it possible to determine the inhibitory activity (IC₅₀) in the above mentioned Berichrom® test:
—(SEQ ID No. 87) IC₅₀=19 μM
(SEQ ID No. 88) IC₅₀=˜280 μM

### Example 4

### Expressing and Purifying, from Escherichia Coli, Tridegins which have been Modified by the Replacement of Cysteine Residues

These modified tridegins were produced by site-directed mutagenesis of the cysteines present in the wild-type tridegin. The aim of this mutagenesis was to replace, in a step-wise manner, the cysteine residues which are suitable for forming intermolecular disulfide bridges. The tendency to form disulfide bridges, and the aggregation of the end product which accompanies it, were detected by carrying out appropriate chromatographic analyses of the original transglutaminase inhibitor (SEQ ID No. 1) in the presence or absence of reducing agents (e.g. mercaptoethanol and DTT). Thus, in one gel filtration run (on an Amersham-Pharmacia HiPrep 26/60 Sephacryl S200 HR column; 20 mM sodium phosphate, pH 8.0, 300 mM NaCl; 1 ml/min) the purified, recombinant tridegin polypeptide had approximately an 80% content of multimeric, high molecular weight aggregates. The content of the aggregates was significantly lower (<20%) if the recombinant tridegin was separated in a gel filtration run under reducing conditions, in 1.5 mM DTT, 20 mM sodium phosphate, pH 8.0, 300 mM NaCl.

The mutagenesis was carried out using the QuikChange reagents (from Stratagene) in accordance with the manufacturer's instructions. The oligonucleotides SEQ ID No. 41 to SEQ ID No. 46, and the respective reverse-complementary sequences, were used for the mutagenesis. The DNA sequences of the resulting mutants were checked by sequencing.

Current methods were used to transfer the respective coding sequence, in plasmid pET22b, into the Escherichia coli expression strain Origami® B(DE3) (from Novagen), and the strain was then cultured in liquid LB medium as described above. Expression, purification and determi-nation of the activity were carried out as described in example 1. The inhibitory activities which were measured for the individual mutants are shown in the following table 4. The mutants were named in accordance with the XnY scheme.

In this scheme, X denotes the amino acid which was changed by mutagenesis, while n defines the position of this amino acid in the polypeptide chain and Y denotes the amino acid which is present after the mutagenesis.

TABLE 4

Inhibitory effect on factor XIIIa, in the
Berichrom ® assay, of tridegins which have been
modified by replacement of cysteine residues.

| | Relative | | |
|---|---|---|---|
| Cys | inhibitory | | SEQ ID |
| variant | effect (%) | Oligonucleotide | No. |
| SEQ ID | 100 | | |
| No. 1 for | | | |
| comparison | | | |
| C06A | 72 | 5′-gggttaggaataccttgatgccattctttggcagg- | 41 |
| | | caacagtttcatatg | |
| C18A | 93 | 5′-cattccagatcagccccacaccaggcacgagggt- | 42 |
| | | taggaatac | |
| C20A | 63 | 5′-cattccagatcagccccagcccagcaacgagggt- | 43 |
| | | tagg | |
| C26A | 82 | 5′-gtattggtcttgtgcggcttccagatcagcccc- | 44 |
| | | acaccag | |
| C32A | 86 | 5′-gacattgaggaatgaaggcagcgtattggtcttgtg- | 45 |
| | | cgcattcc | |
| C26A; | 80 | | |
| +C38A | | | |
| C3 8A | | 5′-cagttctgaacgtggacgagcttgaggaatgaagg- | 46 |
| | | cacagtattgg | |

The relative inhibitory effect (%) was determined at a final variant concentration of 5.45 μM. The inhibitory effect of the recombinant wild-type tridegin poly-peptide (at 5.45 μM) was normalized to 100%. The oligonucleotides which were used to produce the abovementioned variants are also specified in the table.

### Example 5

### Improving the Fibrinolytic Activity of Tissue Plasminogen Activator (tPa) and Urokinase in the Presence of Recombinant Tridegin or a Tridegin Fragment

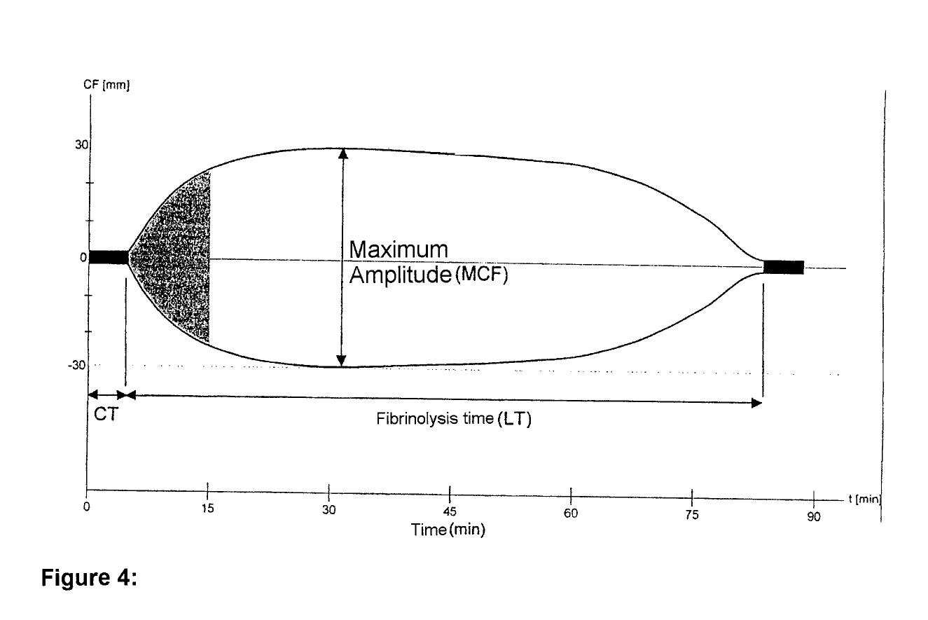

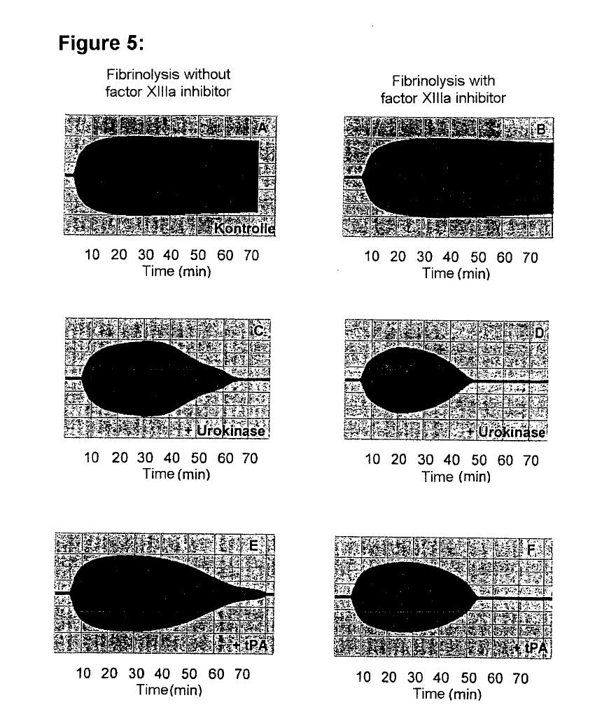

In order to demonstrate the therapeutic potential of the polypeptides according to the invention, blood coagulation and fibrinolysis was measured in whole blood in the presence of recombinant tridegin poly-peptide from E. coli (SEQ ID No. 1, FIG. 5) or tridegin polypeptide from Pichia Pastoris (SEQ ID No. 91, FIG. 11) or tridegin fragments (SEQ ID No. 25, FIG. 10). To do this, what are termed thrombelasto-grams were plotted. Thrombelastography is a current method for measuring coagulation and fibrinolysis. The method measures the change in the viscosity of the blood by the change in the resistance to rotation of a plunger which is present in the blood (Calatzis et al., 2000). FIG. 4 shows the typical phases of coagulation and fibrinolysis in a thrombelastogram. Thrombelastograms quantify important parameters of haemostasis:
clotting time (time between the initiation of coagulation and the beginning of a measurable change in the viscosity of the blood, CT)
stability of the thrombus (maximum amplitude, maximum clot firmness, MCF)
fibrinolysis time (time between the beginning of a measurable change in blood viscosity and achievement of the starting value prior to the clotting, lysis time, LT).

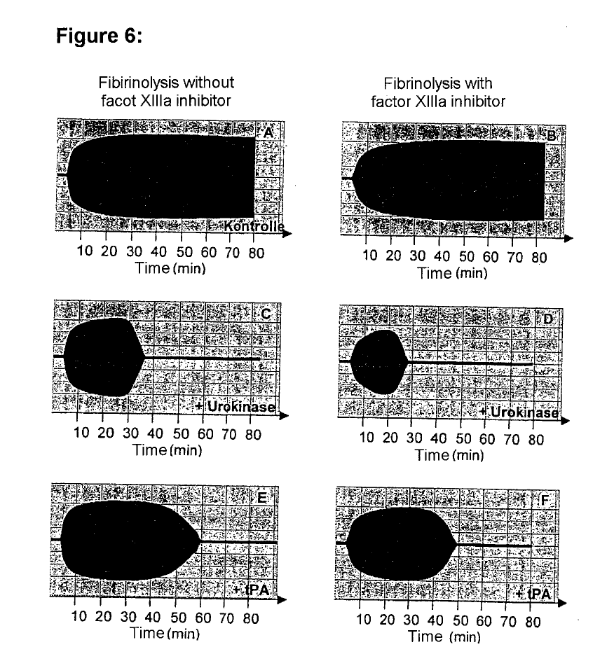

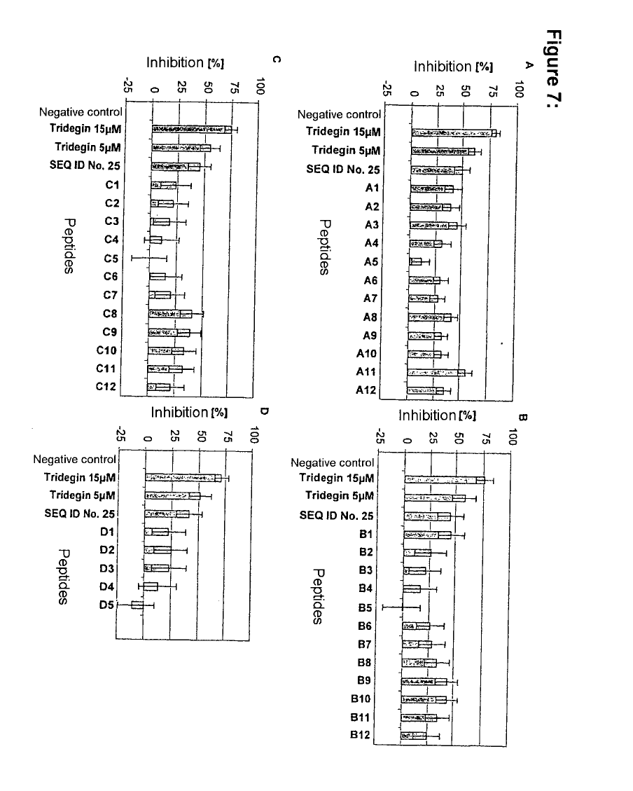

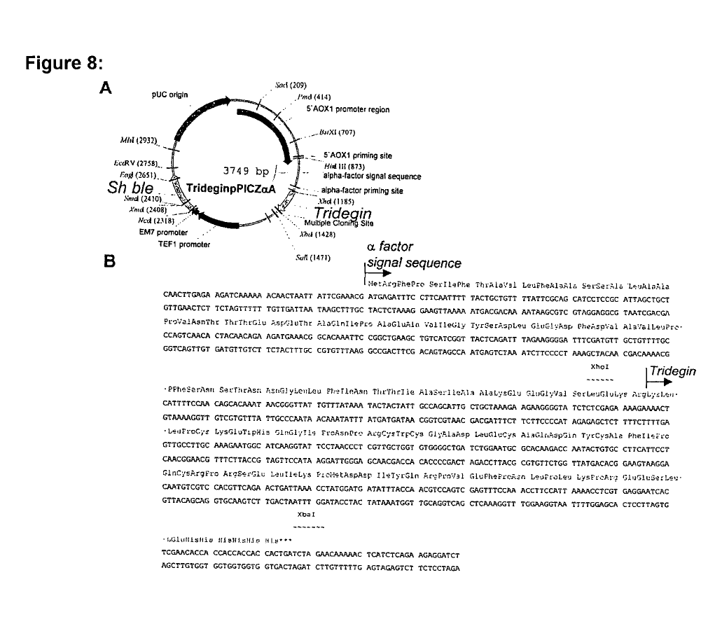

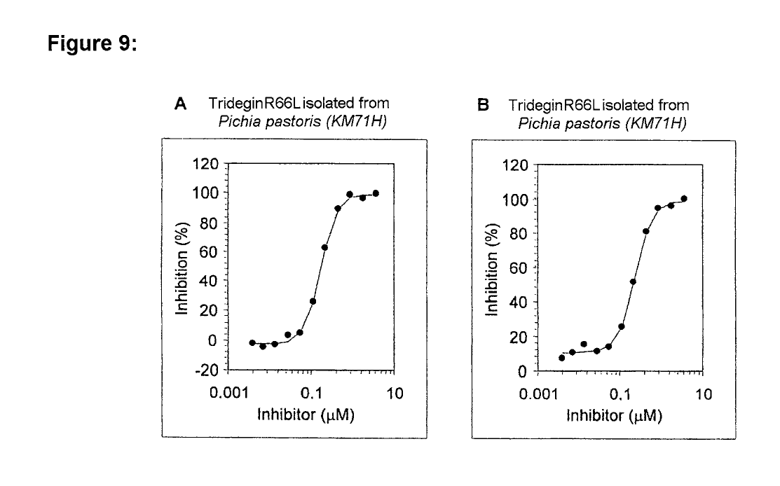

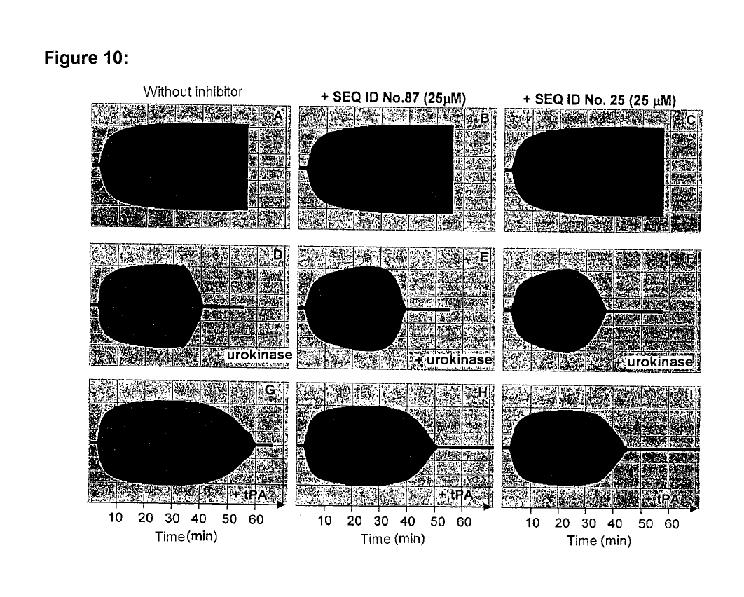

The ROTEG® instrument supplied by Pentapharm GmbH, Munich, was used for the measurements which are shown. FIGS. 5 and 6 show the thrombelastograms of whole citrate blood after the addition of Ca²⁺ (Starteg reagent, from Pentapharm GmbH) and thromboplastin phospholipid (Integ reagent, from Pentapharm GmbH). These reagents are used to induce coagulation. Various experiments were carried out for the purpose of demonstrating the synergic effect, according to the invention, of conventional fibrinolytic agents, which are used in thrombosis therapy, and recombinant tridegin polypeptide and modified tridegins. The thrombelastograms show that the recombinant tridegin polypeptide, and a modified tridegin, accelerate and improve the fibrinolysis which is brought about by the fibrinolytic agents tissue plasminogen activator (tPA) and urokinase, which are selected as an example. This is made clear by

a) the lower amplitude (lower stability of the thrombus) and
b) the increase in the rate of fibrinolysis.

In addition, the extension of the clotting time (CT) from 190 seconds, in the absence of a transglutaminase inhibitor, to 380 seconds, in the presence of the recombinant wild-type tridegin polypeptide from E. coli (SEQ ID No: 1) or of a modified tridegin, shows that the recombinant tridegin polypeptide and a modified tridegin also inhibit blood coagulation (cf. FIGS. 5 A and B).

### Example 6

### Expressing and Purifying Recombinant Tridegin Polypeptide (Encoded by Seq Id No. 90) from Pichia Pastoris

The expression plasmid trideginpPICZαA (based on the expression vector pPICZαA, Invitrogen), which contains the sequence encoding the recombinant tridegin, is depicted in FIG. 8.

SEQ ID No. 90:
atgagatttccttcaatttttactgctgttttattcgcagcatcctccgcattagctgctc-
cagtcaacactacaacagaagatgaaacggcacaaattccggctgaagctgtcatcggt-
tactcagatttagaaggggatttcgatgttgctgttttgccattttccaacagcacaaa-
taacgggttattgtttataaatactactattgccagcattgctgctaaagaagaagggg-
tatctctcgagaaaagaaaactgttgccttgcaaagaatggcatcaaggtattcctaaccc-
tcgttgctggtgtggggctgatctggaatgcgcacaagaccaatactgtgccttcattcct-
caatgtcgtccacgttcagaactgattaaacctatggatgatatttaccaacgtccagt-
cgagtttccaaaccttccattaaaacctcgtgaggaatcactcgaacaccaccaccaccaccactga

By being fused with the alpha factor signal peptide, the tridegin can be secreted into the culture medium. Current methods were used to transfer the plasmid into the Pichia pastoris strains KM71H and SMD1168. Clones containing a stably integrated tridegin sequence were chosen by selecting zeocin-resistant clones and then using the customary method of polymerase chain reaction (PCR) to detect the tridegin DNA sequence.

The resulting clones were cultured (30° C.), for approx. 16-24 hours and while being shaken, as single colonies in 100 ml of BMGH (1% yeast extract; 2% peptone; 100 mM K-phosphate, pH 6; 1.34% yeast nitrogen base; 4×10⁻⁵% biotin; 1% glycerol). The cells were centrifuged down (3000×g, 5 min) and resuspended in 20-30 ml of BMMH (1% yeast extract; 2% peptone; 100 mM K-phosphate, pH 6; 1.34% yeast nitrogen base; 4×10⁻⁵% biotin; 0.5% methanol) and once again incubated at 30° C. while being shaken. After 24 hours, methanol (final concentration, 0.5%) was added. After a further 24 hours, the cells were centrifuged down as described above. The culture supernatant was either processed directly or stored at −70° C. The tridegin polypeptide was detected by means of SDS polyacrylamide gel electrophoresis and Coomassie brilliant blue stain. The correct processing of the signal peptide was confirmed by subjecting the poly-peptide to N-terminal sequencing (Edman degradation, Toplab GmbH). However, the completely expressed tridegin polypeptide, containing the 6 C-terminal histidine residues, was only detected in small quantities using a current Western blotting method (antibody against 5 consecutive histidine residues, Quiagen AG). The missing C-terminal protein sequence GluGluSerLeuGluH is HisHisHis-His His was found by means of mass spectroscopy (MALDI, Toplab GmbH).

The main product, a tridegin polypeptide without the 11 C-terminal residues, was purified using the following method. The culture supernatant was treated with (NH₄)₂SO₄ at the rate of 10 g of (NH₄)₂SO₄ per 25 ml of culture supernatant. The resulting precipitate was centrifuged down and dissolved in 20 mM CHES, pH 9; this solution was then dialyzed against 20 mM CHES, pH 9. The sample was then loaded onto a Sepharose Q (25 ml) or Resource Q (1 ml) ion exchange column (both obtained from Amersham Biosciences) (flow rate, 1-4 ml/min). The column was eluted with a gradient of 20 mM CHES, pH 9, against 20 mM CHES, 1 M NaCl, pH 9. The fractions which were collected during the elution were fractionated by means of SDS polyacrylamide gel electrophoresis and stained with Coomassie brilliant blue. In this way, tridegin polypeptide-containing fractions were identified. These fractions were combined and dialyzed against PBS (0.2 g of KCl/l, 0.2 g of KH₂PO₄/l, 8 g of NaCl/l, 1.15 g of Na₂HPO₄/l, pH 7.2) and concentrated by means of ultrafiltration (Centricon YM-3, Amicon). FIG. 9 shows an FXIIIa inhibition curve which was obtained using the tridegin samples which were isolated from Pichia pastoris. The above-described Berichrom assay was used for determining the activity.

### Example 7

### Expressing and Purifying Modified Recombinant Tridegin Polypeptide (Encoded by SEQ ID No. 91) from Pichia Pastoris

The tridegin polypeptide which was expressed and purified in example 6 lacks 6 C-terminal histidines. These histidines were presumably cleaved off by a protease. A current site-directed mutagenesis method was therefore used to alter the coding DNA sequence in order to inhibit the protease digestion of the encoded tridegin polypeptide. In connection with this, the expression plasmid pPICZalphaA-trideginR66L was generated (tridegin sequence, SEQ ID 91). In the tridegin poly-peptide sequence, an arginine residue at the C terminus was replaced with leucine.

SEQ ID 91:
atgagatttccttcaatttttactgctgttttattcgcagcatcctccgcattagctgctc-
cagtcaacactacaacagaagatgaaacggcacaaattccggctgaagctgtcatcggt-
tactcagatttagaaggggatttcgatgttgctgttttgccattttccaacagcacaaa-
taacgggttattgtttataaatactactattgccagcattgctgctaaagaagaagggg-
tatctctcgagaaaagaaaactgttgccttgcaaagaatggcatcaaggtattcctaaccc-
tcgttgctggtgtggggctgatctggaatgcgcacaagaccaatactgtgccttcattcct-
caatgtcgtccacgttcagaactgattaaacctatggatgatatttaccaacgtccagt-
cgagtttccaaaccttccattaaaacctctggaggaatcactcgaacaccaccaccaccaccactga

The expression plasmid was used for expressing tridegin polypeptide as described in example 6. In connection with this, it was found that it was possible to use the above-described Western blotting method to detect the complete protein sequence, containing the C-terminal histidines, in a surprisingly high yield.

This product was purified using the following method. 1 M Na phosphate, pH 8, was added to the culture super-natant until a pH of 7.4 was reached. The culture supernatant was then diluted 1:1 with buffer A (50 mM NaH₂PO₄, pH 8, 300 mM NaCl, 10 mM imidazole) and passed through a nickel-NTA column (Quiagen) as described in example 1. The column was then washed with buffer A and eluted using elution buffer (50 mM NaH₂PO₄, 300 mM NaCl, 250 mM imidazole, pH 8). Fractions were collected and tested, by means of SDS-PAGE, for the presence of the transglutaminase inhibitor. Fractions which contain the tridegin polypeptide were combined and dialyzed against PBS (0.2 g of KCl/l, 0.2 g of KH₂PO₄/l, 8 g of NaCl/l, 1.15 g of Na₂HPO₄/l , pH 7.2). FIG. 9 shows an FXIIIa inhibition curve which was obtained using these tridegin samples which were isolated in this way from Pichia pastoris. The above-described Berichrom® assay was used for determining the activity.

### Example 8

The influence of the tridegin polypeptide-derived peptides on blood coagulation and fibrinolysis was also measured (FIG. 10). The peptides (SEQ ID No. 25 and SEQ ID No. 88) were dissolved in PBS and diluted. Whole blood (stored at 4° C. for 24 h) was used for the measurements shown in FIG. 10.

The influence of recombinant, purified tridegin poly-peptide isolated from Pichia pastoris on blood coagulation and fibrinolysis is shown in FIG. 11. The tridegin polypeptide (tridegin R66L, encoded by SEQ ID No. 91) was used for these measurements. It reduces the maximum amplitude of the thrombelastograms (i.e. the stability of the clot, MCF) and, in addition, shortens the fibrinolysis time (LT) in the presence of tPA or urokinase.

1. A polypeptide comprising the amino acid sequence of SEQ ID NO: 1, characterized in that it contains at least one modification selected from a replacement of at least one cysteine with another amino acid and/or from a replacement of at least one of the following amino acids—Lys2, Lys7, His10, Gly12, Leu24, Tyr31, Phe34, Arg39, Ile45, Met48, Asp50, Pro55, Phe58, Asn60, Pro65—with another amino acid and/or from a deletion of the N and C termini, with the remaining polypeptide containing at least the amino acid sequence DDIYQRXVXFPXLPL (SEQ ID NO. 89), and/or from a covalent linkage to polyethylene glycol.

2. The polypeptide as claimed in claim 1, wherein the polypeptide is a fusion protein.

3. The polypeptide as claimed in claim 2, wherein the fusion protein contains a tag which is used for purifying the fusion protein.

4. The polypeptide as claimed in claim 3, wherein the tag contains at least five consecutive histidines.

5. The polypeptide as claimed in claim 1, wherein the polypeptide is prepared by a recombinant method or a method of peptide chemistry.

6. The polypeptide as claimed in claim 1, wherein the polypeptide is a transglutaminase inhibitor.

7. A method for preventing or treating thrombosis, the method comprising administering to a subject in need thereof a polypeptide as claimed in claim 1.

8. A pharmaceutical composition comprising a polypeptide as claimed in claim 1.

9. The pharmaceutical composition as claimed in claim 8, wherein the composition comprises at least one galenic adjuvant.

10. The pharmaceutical composition as claimed in claim 8, the composition further comprising at least one additional pharmaceutically active compound.

11. The pharmaceutical composition as claimed in claim 10, characterized in that the additional active compound is an anticoagulant, preferably an inhibitor of thrombin and factor Xa and/or an inhibitor of blood platelet aggregation.

12. The pharmaceutical composition as claimed in claim 8 and, where appropriate, an additional pharmaceutically active compound in arbitrary combination with acetylsalicylic acid, heparin, low molecular weight heparin, heparinoid, hirudin, bivalirudin, melagatran, abciximab, eptifibabide, tissue plasminogen activator (tPA), streptokinase, staphylokinase, urokinase, eminase, hementin and/or plasmin.


SEQUENCE LISTING (102 sequences)

SEQ ID NO: 1 - protein, 68 aa, Artificial Sequence
  Synthetic
MKLLPCKEWHQGIPNPRCWCGADLECAQDQYCAFIPQCRPRSELIKPMDDIYQRPVEFPNLPLKPREE

SEQ ID NO: 2 - protein, 20 aa, Artificial Sequence
  Synthetic
MKLLPCKEWHQGIPNPRCWC

SEQ ID NO: 3 - protein, 20 aa, Artificial Sequence
  Synthetic
LLPCKEWHQGIPNPRCWCGA

SEQ ID NO: 4 - protein, 20 aa, Artificial Sequence
  Synthetic
PCKEWHQGIPNPRCWCGADL

SEQ ID NO: 5 - protein, 20 aa, Artificial Sequence
  Synthetic
KEWHQGIPNPRCWCGADLEC

SEQ ID NO: 6 - protein, 20 aa, Artificial Sequence
  Synthetic
WHQGIPNPRCWCGADLECAQ

SEQ ID NO: 7 - protein, 20 aa, Artificial Sequence
  Synthetic
QGIPNPRCWCGADLECAQDQ

SEQ ID NO: 8 - protein, 20 aa, Artificial Sequence
  Synthetic
IPNPRCWCGADLECAQDQYC

SEQ ID NO: 9 - protein, 20 aa, Artificial Sequence
  Synthetic
NPRCWCGADLECAQDQYCAF

SEQ ID NO: 10 - protein, 20 aa, Artificial Sequence
  Synthetic
RCWCGADLECAQDQYCAFIP

SEQ ID NO: 11 - protein, 20 aa, Artificial Sequence
  Synthetic
WCGADLECAQDQYCAFEPQC

SEQ ID NO: 12 - protein, 20 aa, Artificial Sequence
  Synthetic
GADLECAQDQYCAFIPQCRP

SEQ ID NO: 13 - protein, 20 aa, Artificial Sequence
  Synthetic
DLECAQDQYCAFIPQCRPRS

SEQ ID NO: 14 - protein, 20 aa, Artificial Sequence
  Synthetic
ECAQDQYCAFIPQCRPRSEL

SEQ ID NO: 15 - protein, 20 aa, Artificial Sequence
  Synthetic
AQDQYCAFIPQCRPRSELIK

SEQ ID NO: 16 - protein, 20 aa, Artificial Sequence
  Synthetic
DQYCAFIPQCRPRSELIKPM

SEQ ID NO: 17 - protein, 20 aa, Artificial Sequence
  Synthetic
YCAFEPQCRPRSELIKPMDD

SEQ ID NO: 18 - protein, 20 aa, Artificial Sequence
  Synthetic
AFIPQCRPRSELIKPMDDIY

SEQ ID NO: 19 - protein, 20 aa, Artificial Sequence
  Synthetic
IPQCRPRSELIKPMDDIYQR

SEQ ID NO: 20 - protein, 20 aa, Artificial Sequence
  Synthetic
QCRPRSELIKPMDDIYQRPV

SEQ ID NO: 21 - protein, 20 aa, Artificial Sequence
  Synthetic
RPRSELIKPMDDIYQRPVEF

SEQ ID NO: 22 - protein, 20 aa, Artificial Sequence
  Synthetic
RSELIKPMDDIYQRPVEFPN

SEQ ID NO: 23 - protein, 20 aa, Artificial Sequence
  Synthetic
ELIKPMDDIYQRPVEFPNLP

SEQ ID NO: 24 - protein, 20 aa, Artificial Sequence
  Synthetic
IKPMDDIYQRPVEFPNLPLK

SEQ ID NO: 25 - protein, 20 aa, Artificial Sequence
  Synthetic
PMDDIYQRPVEFPNLPLKPR

SEQ ID NO: 26 - protein, 20 aa, Artificial Sequence
  Synthetic
DDIYQRPVEFPNLPLKPREE

SEQ ID NO: 27 - DNA, 54 bp, Artificial Sequence
  Synthetic
ctcgactggacgttggtaaatagcatccataggtttaatcagttctgaacgtgg

SEQ ID NO: 28 - DNA, 43 bp, Artificial Sequence
  Synthetic
gtggacgacattgaggaatggcggcacagtattggtcttgtgc

SEQ ID NO: 29 - DNA, 54 bp, Artificial Sequence
  Synthetic
cgaggttttaatggaaggtttggagcctcgactggacgttggtaaatatcatcc

SEQ ID NO: 30 - DNA, 46 bp, Artificial Sequence
  Synthetic
cttgcaaagaatggcatcaagctattcctaaccctcgttgctggtg

SEQ ID NO: 31 - DNA, 44 bp, Artificial Sequence
  Synthetic
cgagggttaggaataccttgagcccattctttgcaaggcaacag

SEQ ID NO: 32 - DNA, 54 bp, Artificial Sequence
  Synthetic
ttggtaaatatcatccataggtttagccagttctgaacgtggacgacattgagg

SEQ ID NO: 33 - DNA, 50 bp, Artificial Sequence
  Synthetic
ccttgatgccattctttgcaaggcaacagtgccatatgtatatctccttc

SEQ ID NO: 34 - DNA, 50 bp, Artificial Sequence
  Synthetic
catatgaaactgttgccttgcgcagaatggcatcaaggtattcctaaccc

SEQ ID NO: 35 - DNA, 40 bp, Artificial Sequence
  Synthetic
gtattggtcttgtgcgcattccgcatcagccccacaccag

SEQ ID NO: 36 - DNA, 55 bp, Artificial Sequence
  Synthetic
cgactggacgttggtaaatatcatccgcaggtttaatcagttctgaacgtggacg

SEQ ID NO: 37 - DNA, 43 bp, Artificial Sequence
  Synthetic
cctcacgaggttttaatggaagggctggaaactcgactggacg

SEQ ID NO: 38 - DNA, 45 bp, Artificial Sequence
  Synthetic
gtggtgctcgagtgattcctcacgagcttttaatggaaggtttgg

SEQ ID NO: 39 - DNA, 53 bp, Artificial Sequence
  Synthetic
ggtttaatcagttctgaacgtggagcacattgaggaatgaaggcacagtattg

SEQ ID NO: 40 - DNA, 43 bp, Artificial Sequence
  Synthetic
gaggaatgaaggcacaagcttggtcttgtgcgcattccagatc

SEQ ID NO: 41 - DNA, 50 bp, Artificial Sequence
  Synthetic
gggttaggaataccttgatgccattctttggcaggcaacagtttcatatg

SEQ ID NO: 42 - DNA, 43 bp, Artificial Sequence
  Synthetic
cattccagatcagccccacaccaggcacgagggttaggaatac

SEQ ID NO: 43 - DNA, 38 bp, Artificial Sequence
  Synthetic
cattccagatcagccccagcccagcaacgagggttagg

SEQ ID NO: 44 - DNA, 40 bp, Artificial Sequence
  Synthetic
gtattggtcttgtgcggcttccagatcagccccacaccag

SEQ ID NO: 45 - DNA, 44 bp, Artificial Sequence
  Synthetic
gacattgaggaatgaaggcagcgtattggtcttgtgcgcattcc

SEQ ID NO: 46 - DNA, 46 bp, Artificial Sequence
  Synthetic
cagttctgaacgtggacgagcttgaggaatgaaggcacagtattgg

SEQ ID NO: 47 - protein, 16 aa, Artificial Sequence
  Synthetic
DDIYGRPVEFPNLPLK

SEQ ID NO: 48 - protein, 19 aa, Artificial Sequence
  Synthetic
MDDIYQRPVEFPNLPLKPR

SEQ ID NO: 49 - protein, 18 aa, Artificial Sequence
  Synthetic
DDIYQRPVEFPNLPLKPR

SEQ ID NO: 50 - protein, 17 aa, Artificial Sequence
  Synthetic
DIYQRPVEFPNLPLKPR

SEQ ID NO: 51 - protein, 16 aa, Artificial Sequence
  Synthetic
IYQRPVEFPNLPLKPR

SEQ ID NO: 52 - protein, 15 aa, Artificial Sequence
  Synthetic
YQRPVEFPNLPLKPR

SEQ ID NO: 53 - protein, 14 aa, Artificial Sequence
  Synthetic
QRPVEFPNLPLKPR

SEQ ID NO: 54 - protein, 13 aa, Artificial Sequence
  Synthetic
RPVEFPNLPLKPR

SEQ ID NO: 55 - protein, 12 aa, Artificial Sequence
  Synthetic
PVEFPNLPLKPR

SEQ ID NO: 56 - protein, 11 aa, Artificial Sequence
  Synthetic
VEFPNLPLKPR

SEQ ID NO: 57 - protein, 10 aa, Artificial Sequence
  Synthetic
EFPNLPLKPR

SEQ ID NO: 58 - protein, 19 aa, Artificial Sequence
  Synthetic
PMDDIYQRPVEFPNLPLKP

SEQ ID NO: 59 - protein, 18 aa, Artificial Sequence
  Synthetic
PMDDIYQRPVEFPNLPLK

SEQ ID NO: 60 - protein, 17 aa, Artificial Sequence
  Synthetic
PMDDIYQRPVEFPNLPL

SEQ ID NO: 61 - protein, 16 aa, Artificial Sequence
  Synthetic
PMDDIYQRPVEFPNLP

SEQ ID NO: 62 - protein, 15 aa, Artificial Sequence
  Synthetic
PMDDIYQRPVEFPNL

SEQ ID NO: 63 - protein, 14 aa, Artificial Sequence
  Synthetic
PMDDIYQRPVEFPN

SEQ ID NO: 64 - protein, 13 aa, Artificial Sequence
  Synthetic
PMDDIYQRPVEFP

SEQ ID NO: 65 - protein, 11 aa, Artificial Sequence
  Synthetic
PMDDIYQRPVE

SEQ ID NO: 66 - protein, 10 aa, Artificial Sequence
  Synthetic
PMDDIYQRPV

SEQ ID NO: 67 - protein, 20 aa, Artificial Sequence
  Synthetic
PMDDIYQRPVEFPNLPLKPA

SEQ ID NO: 68 - protein, 20 aa, Artificial Sequence
  Synthetic
PMDDIYQRPVEFPNLPLKAR

SEQ ID NO: 69 - protein, 20 aa, Artificial Sequence
  Synthetic
PMDDIYQRPVEFPNLPLAPR

SEQ ID NO: 70 - protein, 20 aa, Artificial Sequence
  Synthetic
PMDDIYQRPVEFPNLPAKPR

SEQ ID NO: 71 - protein, 20 aa, Artificial Sequence
  Synthetic
PMDDIYQRPVEFPNLALKPR

SEQ ID NO: 72 - protein, 20 aa, Artificial Sequence
  Synthetic
PMDDIYQRPVEFPNAPLKPR

SEQ ID NO: 73 - protein, 20 aa, Artificial Sequence
  Synthetic
PMDDIYQRPVEFPALPLKPR

SEQ ID NO: 74 - protein, 20 aa, Artificial Sequence
  Synthetic
PMDDIYQRPVEFANLPLKPR

SEQ ID NO: 75 - protein, 20 aa, Artificial Sequence
  Synthetic
PMDDIYQRPVEAPNLPLKPR

SEQ ID NO: 76 - protein, 20 aa, Artificial Sequence
  Synthetic
PMDDIYQRPVAFPNLPLKPR

SEQ ID NO: 77 - protein, 20 aa, Artificial Sequence
  Synthetic
PMDDIYQRPAEFPNLPLKPR

SEQ ID NO: 78 - protein, 20 aa, Artificial Sequence
  Synthetic
PMDDIYQRAVEFPNLPLKPR

SEQ ID NO: 79 - protein, 20 aa, Artificial Sequence
  Synthetic
PMDDIYQAPVEFPNLPLKPR

SEQ ID NO: 80 - protein, 20 aa, Artificial Sequence
  Synthetic
PMDDIYARPVEFPNLPLKPR

SEQ ID NO: 81 - protein, 20 aa, Artificial Sequence
  Synthetic
PMDDIAQRPVEFPNLPLKPR

SEQ ID NO: 82 - protein, 20 aa, Artificial Sequence
  Synthetic
PMDDAYQRPVEFPNLPLKPR

SEQ ID NO: 83 - protein, 20 aa, Artificial Sequence
  Synthetic
PMDAIYQRPVEFPNLPLKPR

SEQ ID NO: 84 - protein, 20 aa, Artificial Sequence
  Synthetic
PMADIYQRPVEFPNLPLKPR

SEQ ID NO: 85 - protein, 20 aa, Artificial Sequence
  Synthetic
PADDIYQRPVEFPNLPLKPR

SEQ ID NO: 86 - protein, 20 aa, Artificial Sequence
  Synthetic
AMDDIYQRPVEFPNLPLKPR

SEQ ID NO: 87 - protein, 16 aa, Artificial Sequence
  Synthetic
MDDIYQRPVEFPNLPL

SEQ ID NO: 88 - protein, 14 aa, Artificial Sequence
  Synthetic
DDIYQRPVEFPNLP

SEQ ID NO: 89 - protein, 15 aa, Artificial Sequence
  Synthetic
DDIYQRXVXFPXLPL

SEQ ID NO: 90 - DNA, 486 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagaaaactgttgccttgcaaagaatggcatcaaggtattcctaaccctcgttgctggtgtggggctgatctggaatgcgcacaagaccaatactgtgccttcattcctcaatgtcgtccacgttcagaactgattaaacctatggatgatatttaccaacgtccagtcgagtttccaaaccttccattaaaacctcgtgaggaatcactcgaacaccaccaccaccaccactga

SEQ ID NO: 91 - DNA, 486 bp, Artificial Sequence
  Synthetic
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagaaaactgttgccttgcaaagaatggcatcaaggtattcctaaccctcgttgctggtgtggggctgatctggaatgcgcacaagaccaatactgtgccttcattcctcaatgtcgtccacgttcagaactgattaaacctatggatgatatttaccaacgtccagtcgagtttccaaaccttccattaaaacctctggaggaatcactcgaacaccaccaccaccaccactga

SEQ ID NO: 92 - protein, 15 aa, Artificial Sequence
  Synthetic
DDIYGRPVEFPNLPL

SEQ ID NO: 93 - protein, 15 aa, Artificial Sequence
  Synthetic
DDIYQRPVEFPNLPL

SEQ ID NO: 94 - protein, 16 aa, Artificial Sequence
  Synthetic
DDIYGRPVEFPNLPLK

SEQ ID NO: 95 - protein, 20 aa, Artificial Sequence
  Synthetic
XXDDIYQRXVXFPXLPLKXX

SEQ ID NO: 96 - protein, 20 aa, Artificial Sequence
  Synthetic
PMXXIYXXPXEXXNXXLXPR

SEQ ID NO: 97 - protein, 20 aa, Artificial Sequence
  Synthetic
PMDDXXQRPVEFPNLPXKPR

SEQ ID NO: 98 - protein, 16 aa, Artificial Sequence
  synthetic
DDIYQRPVEFPNLPLK

SEQ ID NO: 99 - DNA, 234 bp, Artificial Sequence
  SYNTHETIC
tcagtggtggtggtggtggtgctcgagtgattcctcacgaggttttaatggaaggtttggaaactcgactggacgttggtaaatatcatccataggtttaatcagttctgaacgtggacgacattgaggaatgaaggcacagtattggtcttgtgcgcattccagatcagccccacaccagcaacgagggttaggaataccttgatgccattctttgcaaggcaacagtttcat

SEQ ID NO: 100 - DNA, 234 bp, Artificial sequence
  SYNTHETIC
atgaaactgttgccttgcaaagaatggcatcaaggtattcctaaccctcgttgctggtgtggggctgatctggaatgcgcacaagaccaatactgtgccttcattcctcaatgtcgtccacgttcagaactgattaaacctatggatgatatttaccaacgtccagtcgagtttccaaaccttccattaaaacctcgtgaggaatcactcgagcaccaccaccaccaccactga

SEQ ID NO: 101 - DNA, 559 bp, Artificial Sequence
  SYNTHETIC
caacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagaaaactgttgccttgcaaagaatggcatcaaggtattcctaaccctcgttgctggtgtggggctgatctggaatgcgcacaagaccaatactgtgccttcattcctcaatgtcgtccacgttcagaactgattaaacctatggatgatatttaccaacgtccagtcgagtttccaaaccttccattaaaacctcgtgaggaatcactcgaacaccaccaccaccaccactgatctagaacaaaaactcatctcagaagaggatct

SEQ ID NO: 102 - DNA, 559 bp, Artificial Sequence
  SYNTHETIC
agatcctcttctgagatgagtttttgttctagatcagtggtggtggtggtggtgttcgagtgattcctcacgaggttttaatggaaggtttggaaactcgactggacgttggtaaatatcatccataggtttaatcagttctgaacgtggacgacattgaggaatgaaggcacagtattggtcttgtgcgcattccagatcagccccacaccagcaacgagggttaggaataccttgatgccattctttgcaaggcaacagttttcttttctcgagagataccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaacagcagtaaaaattgaaggaaatctcatcgtttcgaataattagttgttttttgatcttctcaagttg